(19) United States
(12) Patent Application Publication
(10) Pub. No.: US 2017/0100030 A1
(43) Pub. Date: Apr. 13, 2017

(54) SYSTEMS AND METHODS FOR RETINOPATHY WORKFLOW, EVALUATION AND GRADING USING MOBILE DEVICES

(71) Applicant: SocialEyes Corporation, Makawao, HI (US)

(72) Inventors: Nicholas Bedworth, Makawao, HI (US); Sven Bursell, Kanehoe, HI (US); Angel Puerta, Los Altos, CA (US); Justin Wong, Milbrae, CA (US)

(21) Appl. No.: 15/315,881

(22) PCT Filed: Jun. 3, 2015
(86) PCT No.: PCT/US15/34040

§ 371 Date: Dec. 2, 2016

Related U.S. Application Data
(60) Provisional application No. 62/007,223, filed on Jun. 3, 2014.

(51) Int. Cl. A61B 3/00; G06F 3/0488; G06F 3/0482; G06F 3/0484; A61B 3/12; G06F 19/00

(52) U.S. Cl. CPC A61B 3/0025; A61B 3/12; A61B 3/0058; A61B 3/0033; A61B 3/1241; G06F 19/3406; G06F 3/0482; G06F 3/0484; G06F 3/04886; G06F 2203/04803

(57) ABSTRACT

Methods, systems, and computer-readable medium are provided for retinopathy evaluation. A retina image and a first plurality of user input controls representing a categories of features related to retinopathy evaluation, are displayed in a user interface. A selection of a first user input control corresponding to a first selected category of features is received. In response to receiving the selection, a second plurality of user input controls are displayed that represents a first set of features associated with the first selected category of features. A selection of a second user input control is received indicating a first feature appearing in the retina image. Findings are determined based on at least the first selected feature. Information related to a diagnosis is determined based on the one or more findings using the processor of the client mobile device, and the information related to the diagnosis is displayed.

### RELATED APPLICATIONS

This application claims priority to U.S. Provisional Application No. 62/007,223, filed Jun. 3, 2014, the entire contents of which is incorporated herein by reference.

### TECHNICAL FIELD

The present disclosure relates generally to systems and methods for mobile devices, in particular, for retinopathy workflow, evaluation and grading.

### BACKGROUND

The recommended standard of care for any person with diabetes includes at least an annual assessment of level of diabetic retinopathy. If this standard of care is achieved, clinical studies have demonstrated that timely treatment for the level of any diabetic retinopathy found can reduce the risk of vision loss to less than 5%. The International Diabetes Federation estimates that over 400 million people today have diabetes worldwide; more than 550 million may be affected by 2030. Less than half these people have access to the recommended retinal evaluations, treatment planning and on-going management. Consequently, diabetes is becoming a major contributor to otherwise preventable blindness in the world today. Cost-effective systems for retinal imaging, evaluation and management that could perform at this level of scale—namely the entire population of persons with diabetes—would have an impact on reducing unnecessary visual impairment and blindness in the world.

Conventional systems for manual retinopathy evaluation and grading of retinal images commonly require the use of multiple, high-resolution, large display screens so that the user, typically an ophthalmologist, can simultaneously view all of the required input data fields, as well as one or more retinal images. Such systems commonly use a visually-cluttered, form-like interface for entry of all relevant grading information into a permanent evaluation record. The conventional systems typically require a keyboard and mouse to enter the required information into the form. Because the single form-type interface requires a large area for viewing all of the required input data fields simultaneously, the retina images being reviewed are often displayed in a separate screen from that displaying the form, or in a separate window from the window displaying the form. The form-like interface for entry of relevant retinopathy information is not designed for ease of use, accuracy or efficiency; the visual complexity and clutter causes the user to waste time determining whether information has been entered correctly in all required input fields. The form-like interface is usually poorly-designed from a human-factors perspective, and typically represents transferring a paper form onto the computer display screen.

### BRIEF DESCRIPTION OF THE DRAWINGS

Some embodiments are illustrated by way of example in the accompanying drawings and should not be considered as a limitation of the invention:

FIG. 1 is a block diagram illustrating a mobile client device, according to an example embodiment;

FIG. 2 is a block diagram showing a retinopathy workflow, evaluation, and grading system implemented in modules of an application, according to an example embodiment;

FIG. 3 is a flowchart showing a method for guiding a user through a retinopathy evaluation of a retina image, according to an example embodiment;

FIG. 4 is a schematic of a user interface screen for an example system and method implemented as a retinopathy workflow, evaluation and grading application, according to an example embodiment;

FIG. 5 is an example user interface screen displaying a gallery of images, according to an example embodiment;

FIG. 6 is an example user interface screen displaying a retina image and control buttons to guide a user through a retinopathy workflow, evaluation and grading system, according to an example embodiment;

FIG. 7 is an example user interface screen displaying a retina image as a red-free image, according to an example embodiment;

FIG. 8 is an example user interface screen displaying buttons corresponding to a selected category “HMA” to guide a user through a retinopathy workflow, evaluation and grading system, according to an example embodiment;

FIG. 9 is an example user interface screen displaying a selected button “<2A, 1-4Q” corresponding to a selected category “HMA” to guide a user through a retinopathy workflow, evaluation and grading system, according to an example embodiment;

FIG. 10 is an example user interface screen illustrating that information has been received for a specific category “HMA” to guide a user through a retinopathy workflow, evaluation and grading system, according to an example embodiment;

FIG. 11 is an example user interface screen displaying buttons corresponding to a selected category “VB” to guide a user through a retinopathy workflow, evaluation and grading system, according to an example embodiment;

FIG. 12 is an example user interface screen displaying a selected button “>6B, 2-4Q” corresponding to a selected category “VB” to guide a user through a retinopathy workflow, evaluation and grading system, according to an example embodiment;

FIG. 13 is an example user interface screen illustrating that information has been received for a specific category “VB” to guide a user through a retinopathy workflow, evaluation and grading system, according to an example embodiment;

FIG. 14 is an example user interface screen displaying buttons corresponding to a selected category “IRMA” to guide a user through a retinopathy workflow, evaluation and grading system, according to an example embodiment;

FIG. 15 is an example user interface screen displaying a selected button “<8A, 1-3Q” corresponding to a selected category “IRMA” to guide a user through a retinopathy workflow, evaluation and grading system, according to an example embodiment;

FIG. 16 is an example user interface screen illustrating that information has been received for a specific category “IRMA” to guide a user through a retinopathy workflow, evaluation and grading system, according to an example embodiment;

FIG. 17 is an example user interface screen displaying buttons corresponding to a selected category “HE” to guide a user through a retinopathy workflow, evaluation and grading system, according to an example embodiment;

FIG. 18 is an example user interface screen displaying a selected button “500μ” and a marker corresponding to a selected category “HE” to guide a user through a retinopathy workflow, evaluation and grading system, according to an example embodiment;

FIG. 19 is an example user interface screen displaying a marker as moved by a user to indicate a fovea in the retina image and a selected button “<500μ from fovea” corresponding to a selected category “HE” to guide a user through a retinopathy workflow, evaluation and grading system, according to an example embodiment;

FIG. 20 is an example user interface screen illustrating that information has been received for a specific category “HE” to guide a user through a retinopathy workflow, evaluation and grading system, according to an example embodiment;

FIG. 21 is an example user interface screen displaying additional buttons corresponding to categories to guide a user through a retinopathy workflow, evaluation and grading system, according to an example embodiment;

FIG. 22 is an example user interface screen displaying buttons corresponding to a selected category “NVD” to guide a user through a retinopathy workflow, evaluation and grading system, according to an example embodiment;

FIG. 23 is an example user interface screen displaying a selected button “present” corresponding to a selected category “NVD” to guide a user through a retinopathy workflow, evaluation and grading system, according to an example embodiment;

FIG. 24 is an example user interface screen illustrating that information has been received for a specific category “NVD” to guide a user through a retinopathy workflow, evaluation and grading system, according to an example embodiment;

FIG. 25 is an example user interface screen displaying buttons corresponding to a selected category “NVE” to guide a user through a retinopathy workflow, evaluation and grading system, according to an example embodiment;

FIG. 26 is an example user interface screen displaying a selected button “present” corresponding to a selected category “NVE” to guide a user through a retinopathy workflow, evaluation and grading system, according to an example embodiment;

FIG. 27 is an example user interface screen illustrating that information has been received for a specific category “NVE” to guide a user through a retinopathy workflow, evaluation and grading system, according to an example embodiment;

FIG. 28 is an example user interface screen displaying buttons corresponding to a selected category “VH” to guide a user through a retinopathy workflow, evaluation and grading system, according to an example embodiment;

FIG. 29 is an example user interface screen displaying a selected button “Present” corresponding to a selected category “VH” to guide a user through a retinopathy workflow, evaluation and grading system, according to an example embodiment;

FIG. 30 is an example user interface screen illustrating that information has been received for a specific category “VH” to guide a user through a retinopathy workflow, evaluation and grading system, according to an example embodiment;

FIG. 31 is an example user interface screen displaying buttons corresponding to a selected category “PVH” to guide a user through a retinopathy workflow, evaluation and grading system, according to an example embodiment;

FIG. 32 is an example user interface screen displaying a selected button “Present” corresponding to a selected category “PVH” to guide a user through a retinopathy workflow, evaluation and grading system, according to an example embodiment;

FIG. 33 is an example user interface screen illustrating that information has been received for a specific category “PVH” to guide a user through a retinopathy workflow, evaluation and grading system, according to an example embodiment;

FIG. 34 is an example user interface screen displaying a summary of the information entered by the user in a retinopathy workflow, evaluation and grading system, according to an example embodiment;

FIG. 35 is an example user interface screen displaying information related to a diagnosis and treatment recommendation based on a retinopathy workflow, evaluation and grading system, according to an example embodiment;

FIG. 36 is an example user interface screen displaying information related to another diagnosis and treatment recommendation based on different information entered into a retinopathy workflow, evaluation and grading system, according to an example embodiment;

FIG. 37 is an example user interface screen displaying a retina image that is zoomed-in, according to an example embodiment;

FIG. 38 is a schematic illustrating an example user interface screen for a retinopathy workflow, evaluation and grading system, according to an example embodiment;

FIG. 39 is a schematic illustrating an example user interface screen displaying image restoration tools available in a retinopathy workflow, evaluation and grading system, according to an example embodiment;

FIG. 40 is a schematic illustrating an example user interface screen including markers on a retina image in a retinopathy workflow, evaluation and grading system, according to an example embodiment;

FIG. 41 illustrates a network diagram depicting a system for retinopathy workflow, evaluation, and grading for mobile devices, according to an example embodiment; and

FIG. 42 is a block diagram of an exemplary computing device that may be used to implement exemplary embodiments of the retinopathy application described herein.

### DETAILED DESCRIPTION OF EXEMPLARY EMBODIMENTS

Embodiments described herein include systems and methods for evaluation of retinopathy in retina images, typically employing a mobile device for the user interface. Some embodiments include a workflow, evaluation and grading application suitable for mobile devices.

Retinopathy comprises several inflammatory or noninflammatory degenerative diseases of the retina. Retinopathy can be caused by persistent or acute damage to the retina of the eye caused by physiological imbalances resulting from ocular and systemic conditions. Ongoing inflammation, vascular changes and remodeling may develop gradually, over long periods of time, with few or no obvious visual symptoms. The patient may not be aware of the existence, let alone the extent of the disease, until more advanced, and difficult to treat, stages have set in. However, changes in the retina associated with the earliest, as well as the later stages of retinopathy, may be detected using techniques that image the retina and assess its condition. Often, retinopathy is an ocular manifestation of the consequences of systemic disease, such as diabetes, as well as cardiovascular disease and hypertension, obesity, and many other chronic as well as acute conditions.

Diabetic retinopathy of the non-proliferative type is characterized by: the presence and extent of microaneurysms, intraretinal punctate hemorrhages, hard exudates (yellow, waxy exudates), venous beading, intraretinal microvascular abnormality (IRMA), cotton-wool spots, and macular edema. The proliferative form of diabetic retinopathy (PDR), is characterized by neovascularization of the retina and optic disk, which may project into the vitreous, proliferation of fibrous tissue, vitreous hemorrhage, and retinal detachment. Macular edema occurs when fluid leaks into the macular region or fovea, which is the part of the eye that provides the sharpest vision. The fluid makes the macula swell, which in turn blurs central vision critical for reading or other fine tasks. While such edema can occur at any stage of diabetic retinopathy, it is more likely to be found as the disease progresses. About half of the people with proliferative retinopathy also have macular edema.

Hypertensive retinopathy can include irregular narrowing of the retinal arterioles; hemorrhages in the nerve fiber layers and the outer plexiform layer; exudates and cotton-wool patches; arteriosclerotic changes; and papilledema. Other types of retinopathy display similar signs. Causes of retinopathy in general include, but are not limited to, diabetes mellitus, arterial hypertension, prematurity, ionizing radiation, direct sunlight exposure, sickle cell anemia, vascular diseases such as retinal vein or artery occlusion, trauma, and blood hyperviscosity.

The more advanced types of retinopathy are proliferative, meaning they rapidly spread throughout the retina. In general, these result from neovascularization, meaning the overgrowth of retinal blood vessels. Angiogenesis, the sprouting of new vessels, is the hallmark precursor that may result in severe vision loss and blindness, particularly if the macula becomes affected. Retinopathy generally requires assessment and diagnosis by an ophthalmologist, and treatments depend on the cause and stage of the disease. These range from diet and lifestyle recommendations to medication, and ultimately surgery of various kinds.

Retinopathy has various stages, each of which may be diagnosed. In the case of diabetes-related retinopathy, several levels can be described in a systematic manner, according to rules and protocols laid out in the Early Treatment Diabetic Retinopathy Study (ETDRS). This protocol defines four stages as follows. The first stage is mild, nonproliferative diabetic retinopathy (NPDR), in which hemorrhages and microaneurysms (HMAs) occur. Microaneurysms are small areas of balloon-like swelling in the retina's tiny blood vessels; hemorrhages are commonly characterized as small, punctate microbleeds. The second stage of NPDR is moderate, nonproliferative diabetic retinopathy, in which, as the disease progresses, there are increasing numbers of HMAs, as well as some occurrences of hard exudates, venous beading, and intraretinal microvascular abnormalities or IRMA. The third stage of NPDR, severe nonproliferative retinopathy, or sight threatening retinopathy, is characterized by the frank occurrence of venous beading and IRMA coupled with extensive HMAs and hard exudates. At this stage, there are occurrences of capillary non-perfusion, in which small capillary blood vessels are blocked, thus depriving areas of the retina from nutrient delivery. These areas of capillary non-perfusion are drivers of proliferative diabetic retinopathy; new, irregular retinal vessel growth occurs in an attempt to increase nutrient delivery to counter the non-perfusion. In short, PDR is characterized by the growth of new retinal vessels in response to lack of nutrient delivery. These vessels are weak, abnormal in many ways, and break readily, which leads to vitreous hemorrhage, fibrous proliferation, and possibly the eventual loss of vision.

During the early stages of NPDR (mild, or moderate), tertiary-level interventions, such as laser treatment or anti-vascular endothelial growth factor (anti-VEGF) injections, are usually not indicated in the absence of macular edema. Early NPDR interventions, such as improved blood sugar management, control of lipid levels and blood pressure, and related strategies can significantly decrease the risk of progression of diabetic retinopathy. Generally, tertiary treatments are considered at the stage of severe NPDR or PDR. The treatment options are panretinal photocoagulation (PRP) or repeated intravitreal injection of anti-VEGF. The recognized standard of care for all people with diabetes is an annual retinal examination, but more frequent and comprehensive dilated eye exams are required in the case of PDR to determine the optimal time to use PRP or anti-VEGF injections.

Detection and assessment of retinopathy in its early stages can give health providers and patients the opportunity to address underlying health conditions, which if treated properly, can slow or arrest the progression of the retinopathy long before it significantly affects the patient's vision. Further, identification of retinopathy in its early stages may allow treatment options that would be less effective than at later stages when, for example, PRP or anti VEGF injections are required. Catching retinopathy early on can also alert health providers and patients to previously unknown contributing health conditions (e.g., renal disease, cardiovascular disease or hypertension). One of the perhaps ironic consequences of the overall worldwide improvement in longevity is that hundreds of millions of persons with diabetes will now live long enough that the consequences of retinopathy may become apparent during their working lives, and certainly will be a problem during retirement and old age.

Some embodiments described herein provide methods and systems for retinopathy evaluation of retina images using mobile and hand-held devices. Some embodiments include a clinical retinopathy workflow, evaluation and grading application that leverages the touch screen interface that many mobile devices currently include. In some embodiments, the retinopathy workflow, evaluation and grading application displays a retinal image in the center of a user interface and provides input mechanisms on the edges of the user interface for the user to enter information. This helps the user to hold and handle the mobile device easily, while simultaneously selecting inputs with his/her thumbs or other fingers. In contrast, conventional grading software and methodologies, in which information is submitted on one or more large, complex forms, are not suitable for hand-held devices. In the usual methodologies, multiple screens or windows display both the data entry form and the retinal image. Further, the input mechanisms on the user interface (e.g., buttons) are sufficiently large “targets” that allow easy location and accurate actuation by touch under adverse conditions, which is not true of the small and cluttered data input areas on a conventional form-like grading interface, the successful operation of which requires high levels of user understanding and dexterity. Some embodiments permit the use of larger buttons because entry of information is based on a guided workflow that does not require simultaneous display of input controls for all required data.

In some embodiments, the methods and systems described herein conform to the Digital Imaging and Communications in Medicine (DICOM) standard for handling, storing, printing, and transmitting information in medical imaging. The DICOM standard has been standardized by the National Electrical Manufacturers Association (NEMA). The text of the DICOM Standard, published by the DICOM Standard Committee, NEMA, 1300 N. 17^{th} Street, Rosslyn, Va. 22209, USA (available at http://medical.nema.org/), is incorporated by reference herein.

The retinopathy system includes a workflow that guides the user through the various decision points required for evaluating and grading retinopathy, and obtains needed information based on user observations and finding for the decision points. These correspond to categories based on the Early Treatment Diabetic Retinopathy Study (ETDRS) classification system in some embodiments. The various possible observations are collected into decision points. Thus, the input tasks required by the user are compartmentalized, which helps in gathering accurate information. This compartmentalization reduces the visual clutter in the display, as compared with prior art systems for retinopathy assessment. The decision points for assessing clinical level of nonproliferative diabetic retinopathy (NPDR) include the categories of hemorrhages and microaneurysms (HMA); venous beading (VB); intraretinal microvascular abnormalities (IRMA); and hard exudates (HE) in some embodiments. The decision points for assessing the level of proliferative diabetic retinopathy (PDR) include new vessels on the optic disc (NVD); new vessels elsewhere (NVE); vitreous hemorrhage (VH); and preretinal vitreous hemorrhage (PVH), in some embodiments. The retinopathy workflow, evaluation, and grading application requires the user to consider each decision point before findings or diagnostic information is provided. In contrast, because conventional, form-based systems display too many text fields on the screen, the user may tend to skip over some fields and/or decision points, which may lead to an inaccurate diagnosis and inappropriate management plan. In such conventional form-based systems verifying correct use of the forms may require external supervision and correction by skilled medical personnel.

The data gathered represent the user's observations of the retinal image, including characteristics of the retina related to features of retinopathy, such as lesions, bulging of the veins, bleeding in any vessels, and the like. Using the retinopathy lesion findings data, the retinopathy application generates diagnostic information (e.g., a diagnosis) and/or standardized treatment recommendations based on the clinical level of retinopathy. The retinopathy application is compact, and displays the decision points and outcomes at user-friendly areas within the user interface. Decision points can also be thought of, or referred to, as collections of categories of features. Further, the retinopathy application has the ability to inform the user which categories of data are incomplete, before any recommendation can be provided.

In some embodiments, the retinopathy workflow, evaluation, and grading application provides a persistent indication that a feature has been selected for a category via a persistent, initial or first change in color (e.g., from gray or blue to green) of buttons corresponding to the selected features and of buttons corresponding to categories for which a feature has been selected, thereby allowing the user to track the selected features and the completed categories via the current color state of the corresponding buttons (e.g., with green indicating selected features or completed categories). The first color change of the feature buttons may be described as the “selected feature” color change when applied to the feature buttons and described as the “completed category” color change when applied to the categories buttons. Additionally, in some embodiments, the retinopathy workflow, evaluation, and grading application indicates which category is presently selected or active via a second color change (e.g., from gray or green to blue) of a button corresponding to the selected or active category of features. This second color change indicates to the user the category to which the presently displayed features belong. This second color change, which may be described as the “active category color change” when applied to the category buttons is temporary and does not persist. For example, if a first category is currently active (e.g., the button color for the category has already changed from gray or green to blue), when a second category is selected, the button color of the active second category will change (e.g., to blue) and the button color of the first category will change to the appropriate color for its state (e.g., gray for if no features have been selected for the category and green if the category has been completed through selection of a feature). Although the first color change is described herein as persistent, it should be noted that the “active category” color change may temporarily override the completed category color change (e.g., selection of a green completed category button will cause it to change color to a blue active category button while the category is active, but it will revert back to its green completed category color when a new category button is selected). The second color change may also indicate active selections other than categories. For example, the second color change may indicate whether the “red free” option for displaying the retina image is selected. As another example, the second color change may indicate whether a “gallery” option for selecting images is displayed. One of ordinary skill in the art will appreciate that visual indications other than a change in color may be used to indicated a change in state of a category or button. For example, the visual indication may be a change in shape, intensity or pattern of the category or button, or changing from static display to blinking or vice versa. Any such appropriate visual indications fall within the scope of embodiments.

Another advantage of some embodiments of the retinopathy workflow, evaluation, and grading application is the placement of various user input controls in the user interface. While holding a hand-held or mobile device, a user's thumb and/or fingers have a limited range of motion for selecting buttons on the device. If a button or interaction element is beyond the immediate reach of the user's thumb or fingers, then he or she has to change his or her grip on the device. For an application such as the retinopathy workflow, evaluation, and grading application where the user needs to enter a substantial amount of information by the selection of buttons, it is time-consuming and inefficient for a user to change his or her grip of the device to reach one or more commonly used buttons. Therefore, in some embodiments, the retinopathy workflow, evaluation, and grading application is configured to display the commonly used input controls for the decision points and outcomes so that they are within the reach of the user's thumbs, thus eliminating the need to adjust the grip of the device during use.

Another advantage of some embodiments over conventional systems is the portability of the retinopathy workflow, evaluation, and grading application. The user merely needs a mobile or hand-held device to use the retinopathy workflow, evaluation, and grading application, instead of a bulky computer workstation or laptop with multiple display screens or tiled display screens. Furthermore, this approach allows a user to view retinal images and related retinal lesion assessment and clinical diagnoses from remote locations, such as a location away from the patient, or a location away from retinal specialist. Due to its portability, high usability, and automated diagnostic capabilities, the retinopathy workflow, evaluation, and grading application can also be used by a relatively unskilled, non-medically-credential user to educate a patient regarding his/her diagnosis and treatment plan. Additionally, because of its portability, a skilled professional (e.g., ophthalmologist) can use the retinopathy workflow, evaluation, and grading application to confirm a previous assessment of a retinal image or adjudicate a previously assessed retinal image for subsequent research studies.

Because it is designed for a hand-held or mobile device, the retinopathy workflow, evaluation and grading system can aid in engaging the patient in his or her treatment plan. Conventional retinopathy grading systems are designed primarily for multi-screen workstations, and as such, are not convenient for engaging a patient at a more remote treatment or screening location. However, the retinopathy evaluation system described herein, allows the user to show the patient at the point and time of contact, his or her retinal image and any lesions that have developed. This can provide a powerful educational intervention moment during which the risk of vision loss as can be seen in that patient's eye images. This visualization can motivate a patient to become more adherent to taking their medications, going to subsequent eye exams and regular clinic visits. The user can easily compare the images from two separate visits for a graphic illustration of any changes in the retina over time and to evaluate the effectiveness of any lifestyle modifications or treatments on progression of retinopathy. Being able to visualize the results, the patient is more likely to engage in his or her treatment plan actively, and is more likely to be diligent in following the recommended treatment plan.

Similarly, the retinopathy evaluation system can be used to verify a diagnosis or treatment recommendation. In some embodiments, the retinopathy evaluation system can be used to verify or adjudicate a diagnosis previously made by the user, or another user, employing the same retinopathy evaluation system, or a similar, compatible retinopathy evaluation system. For example, selected features, and possibly also findings and a diagnosis for one or more retinal images may be made using a retinopathy evaluation system and the results stored. A remotely located user, such as an ophthalmologist, could load the stored results into another similar retinopathy evaluation system to view previously selected features for each categories of features. These stored results, along with the corresponding retinal image and diagnosis information may be used to verify the accuracy of findings previously made for the retinal images. The previously selected features and the stored results may include a digital signature or identification associated with the user who made the initial evaluation and previous selections of the features. In some embodiments, the stored results may further include a date and time stamp, geolocation data and/or biometric tags. Information in the stored results may indicate the circumstances of any evaluations being made and saved in the device or system.

In some embodiments, if a second user (e.g., an ophthalmologist reviewing the prior analysis) finds the previously selected features, diagnosis and treatment recommendation inaccurate, the second user can alter the selected features. In an example embodiment, the alterations may be indicated by a different color highlighting, such as red. Alternatively, or additionally, the second user can make comments within the application to indicate why he or she disagrees with the diagnosis in some embodiments. In some embodiments, the second user may be unable to alter the previously selected features. In some embodiments, the second user's corrections and notes are stored as an addendum to the original diagnosis. The addendum may include the correcting or confirming user's digital signature or identification information. A date and time stamp, as well as additional contextual data, may also be provided with the addendum indicating when and by whom the results were corrected or the diagnosis was confirmed. In this manner, the retinopathy workflow, evaluation and grading system can aid skilled medical and support personnel in verifying retinopathy assessments.

The systems and methods described herein for retinopathy evaluation, grading and workflow are beneficial for use in remote areas where network connectivity is of poor quality. Many rural areas in third-world and developing countries have poor or no access to Internet, and limited access to healthcare. Even under such circumstances, and amongst the enormous populations in any country who are living at “the edge,” meaning beyond the reach of conventional medical services, the user can perform the retinopathy workflow, evaluation and grading application on truly portable, mobile devices, to grade and evaluate retinal images, and provide treatment plans for the patient. The retinopathy workflow, evaluation and grading application may operate essentially “untethered,” meaning independently and not connected, to the Internet or other telecommunication system. This independence eliminates the need of Internet connection and transport of bulky workstations to rural, remote or other hard to reach areas, in order to perform retinopathy assessment. However, in some embodiments, the system is configured such that when internet connectivity is available, all work stored on the mobile device is uploaded and synchronized with a server to provide a permanent record of the patient encounter.

The following description is presented to enable any person skilled in the art to create and use a computer system configuration and related method and article of manufacture for a retinopathy application including workflow, evaluation, and grading tools. Various modifications to the example embodiments will be readily apparent to those skilled in the art, and the generic principles defined herein may be applied to other embodiments and applications without departing from the spirit and scope of the invention. Moreover, in the following description, numerous details are set forth for the purpose of explanation. However, one of ordinary skill in the art will realize that the invention may be practiced without the use of these specific details. In other instances, well-known structures and processes are shown in block diagram form in order not to obscure the description of the invention with unnecessary detail. Thus, the present disclosure is not intended to be limited to the embodiments shown, but is to be accorded the widest scope consistent with the principles and features disclosed herein.

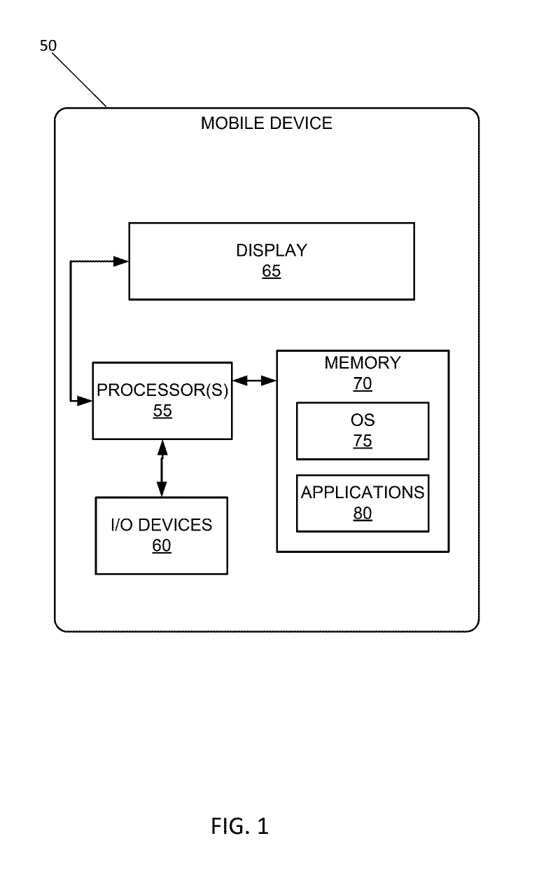

FIG. 1 is a block diagram illustrating a mobile device for implementing systems and methods associated with a retinopathy workflow, evaluation and grading application, according to an example embodiment. In an example embodiment, the mobile device 50 includes one or more processor(s) 55, a memory 70, I/O devices 60, and a display 65. The processor(s) 55 may be any of a variety of different types of commercially available processors suitable for mobile devices (for example, NVIDIA System on a Chip (SoC) multicore processors along with graphics processing units (GPU) devices, such as the TEGRA K1 by NVIDIA Corp., XScale architecture microprocessors, INTEL CORE processors by Intel Corp., INTEL ATOM processors by Intel Corp, INTEL CELERON processors by Intel Corp., INTEL PENTIUM processors, QUALCOMM SNAPDRAGON processors by Qualcomm Inc., ARM® architecture processors by ARM Holdings PLC, Microprocessor without Interlocked Pipeline Stages (MIPS) architecture processors, A series System-on-chip (SoCs) processors by Apple Inc., or another type of processor). The processor(s) 55 may also include one or more graphics processing units (GPUs). The memory 70, such as a Random Access Memory (RAM), a Flash memory, or other type of memory, is accessible to the processor(s) 55. The memory 70 can be adapted to store an operating system (OS) 75, as well as application programs 80, such the retinopathy workflow, evaluation, and grading system described herein. The processor(s) 55 is/are coupled, either directly or via appropriate intermediary hardware, to a display 65 and to one or more input/output (I/O) devices 60, such as a keypad, a touch panel sensor, a microphone, and the like. The mobile device 50 is also capable of establishing wireless communication such as Wi-Fi, Bluetooth and/or Near Field Communication (NFC) connectivity, as well as satellite connectivity and other telecommunication methodologies.

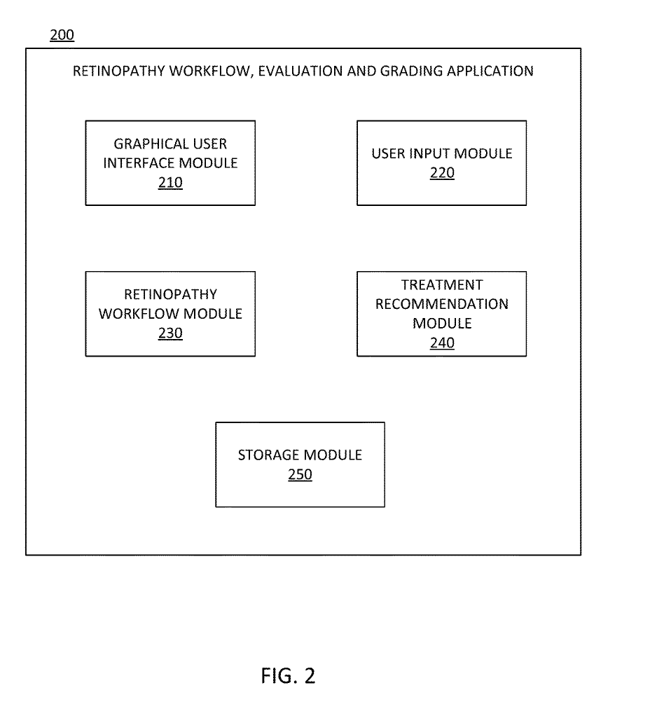

FIG. 2 is a block diagram 200 showing a retinopathy application implemented in modules according to an example embodiment. The modules may be implemented in mobile device 50. The modules may comprise one or more software components, programs, applications, apps or other units of code base or instructions configured to be executed by one or more processors 55 included in mobile device 50. In some embodiments, the modules include a graphical user interface module 210, a user input module 220, a retinopathy workflow module 230, a recommendation module 240, and a storage module 250.

In some embodiments, the modules 210, 220, 230, 240, and 250 may be downloaded from a remote site, for example, from web site associated with a health care provider. In some embodiments, the modules 210, 220, 230, 240, and 250 may be downloaded from an ecommerce site appropriate for the type of computing device. For example, if the client device 110, 115, 120, or 125 comprises an iOS-type device (e.g., IPHONE or IPAD by Apple Inc.), then the modules can be downloaded from ITUNES by Apple Inc. Similarly, if the client device 110, 115, 120 or 125 comprises an Android-type device, then the modules 210, 220, 230, 240, and 250 can be downloaded from the Android Market™ or Google Play Store. If the client device 110, 115, 120, or 125 comprises a Windows® Mobile-type device, then the modules 210, 220, 230, 240, and 250 can be downloaded from Microsoft® Marketplace. The modules 210, 220, 230, 240, and 250 may be packaged as a retinopathy evaluation app. In embodiments for use in areas where internet or wireless service may be unreliable or nonexistent, it may be preferable for all modules to be implemented locally on the client device. Additionally, the modules may include an application programming interface (API) specifying how the various modules of the retinopathy evaluation app interact with each other and with external software applications.

In other embodiments, one or more of modules 210, 220, 230, 240, and 250 may be included in server 135 or database server(s) 140 while other of the modules 210, 220, 230, 240, and 250 are provided in the client devices 110, 115, 120, 125. Although modules 210, 220, 230, 240, and 250 are shown as distinct modules in FIG. 2, it should be understood that modules 210, 220, 230, 240, and 250 may be implemented as fewer or more modules than illustrated. It should be understood that any of modules 210, 220, 230, 240, and 250 may communicate with one or more external components such as databases, servers, database server, or other client devices.

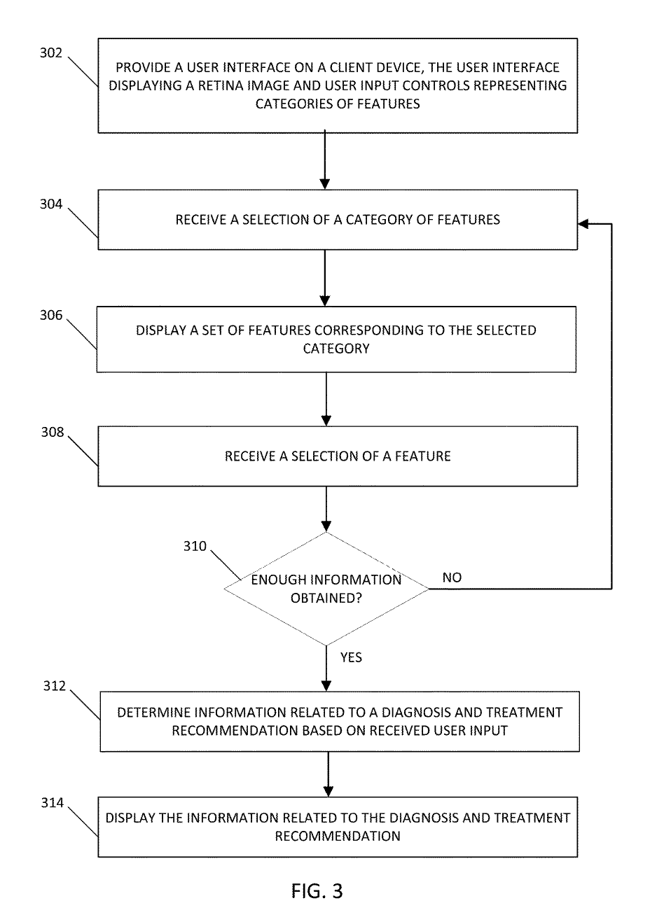

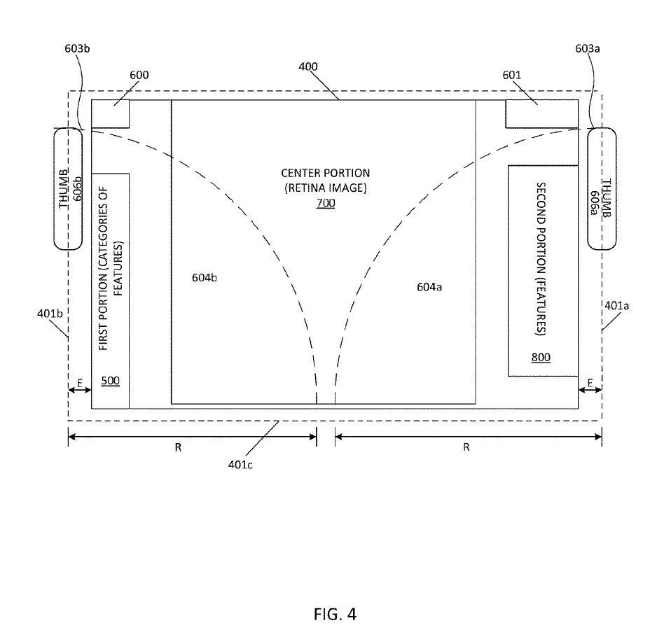

FIG. 3 illustrates an example flow diagram 300 of a method for guiding a user through a retinopathy evaluation of a retina image, according to an embodiment. FIG. 4 is a user interface screen for a retinopathy workflow, evaluation and grading system, according to an example embodiment. A first plurality of user input controls are displayed, which correspond to a plurality of categories of features, on a first side portion of the user interface, illustrated by first portion 500 in screen 400 of FIG. 4. A second plurality of input controls, which correspond to a set of features associated with a selected category of features, is displayed on a second side portion of the user interface, illustrated by second portion 800 in screen 400 of FIG. 4. The retina image is displayed in a center portion of the user interface, illustrated by center portion 700 in screen 400 of FIG. 4. Additional user input controls, such as buttons, are also displayed in the user interface, illustrated by buttons 600, 601 in screen 400 of FIG. 4. Arcs 603a, 603b illustrate a range of motion possible by a user's thumbs 606a, 606b respectively when the user is holding mobile device 50 with one hand or both hands and the mobile device 50 is resting on the user's fingers. For example, a user's thumbs 606a, 606b while holding mobile device 50 may be capable of moving from a vertical position parallel to sides of screen 400 through an arc toward a bottom edge of the screen 400. In some embodiments, the first plurality of user input controls (first portion 500) and/or the second plurality of user input controls (second portion 800) are displayed in screen 400 such that they are within a range of motion of the user's thumbs 606a, 606b. Such placement of buttons in first portion 500 and second portion 800 enables a user to easily select the user input controls and navigate the retinopathy workflow, evaluation and grading application. The user input controls displayed in first portion 500 and second portion 800 are commonly used or used more often than the other user input controls in screen 400, which increases the importance of locating them within a comfortable range of motion of the user's thumb 606.

In some embodiments, the commonly used user input controls are placed in a region of the user interface covered by a first area 604a (enclosed by arc 603a, device edge 401a, and device edge 401c) including a 90 degree arc extending with a radius R of 95 mm from an edge 401a of mobile device and a second area 604b (enclosed by arc 603b, device edge 401b, and device edge 401c) including a 90 degree arc extending with a radius R of 95 mm from an opposite edge 401b of the mobile device. In some embodiments, the commonly used user input controls are placed in a region of the user interface covered by a first area 604a including a 90 degree arc extending with a radius R of 85 mm from an edge 401a of mobile device and a second area 604b including a 90 degree arc extending with a radius R of 85 mm from an opposite edge 401b of the mobile device. In some embodiments, the commonly used user input controls are placed in a region of the user interface covered by a first area 604a including a 90 degree arc extending with a radius R of 75 mm from an edge 401a of mobile device and a second area 604b including a 90 degree arc extending with a radius R of 87 mm from an opposite edge 401b of the mobile device. In some embodiments, the commonly used user input controls are placed in a region of the user interface covered by a first area 604a including a 90 degree arc extending with a radius R of 75 mm from an edge 401a of mobile device and a second area 604b including a 90 degree arc extending with a radius R of 65 mm from an opposite edge 401b of the mobile device.

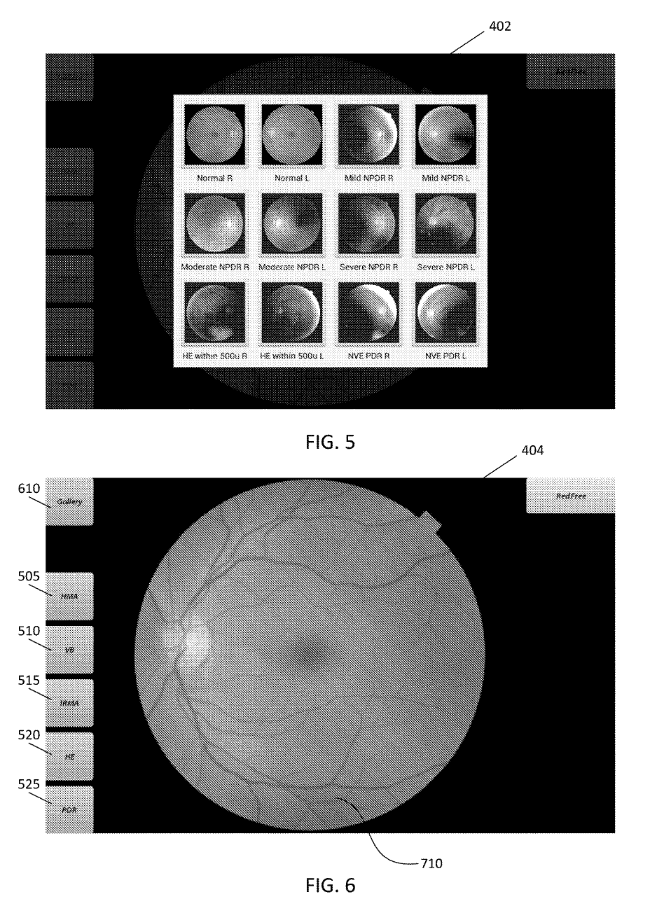

FIGS. 5-37 illustrate examples of user interface screens provided on mobile device 50 associated with an example system and method implemented as a retinopathy workflow, evaluation, and grading application. The discussion below employs the reference numbers used in the mobile client device block diagram of FIG. 1, the retinopathy workflow, evaluation and grading system block diagram of FIG. 3, and the flowchart of the method of FIG. 3, in conjunction with the description of the user interface screens of FIGS. 5-37 to describe one or more embodiments of the retinopathy workflow, evaluation, and grading application. The retinopathy workflow, evaluation, and grading application disclosed herein facilitates evaluation and grading of retinal images by guiding a user through a workflow, and producing diagnosis information and/or a treatment recommendation based on the user input. The user interface provides easy to use mechanisms to aid in entering relevant information for producing diagnosis information and/or a treatment plan.

A user can access the retinopathy evaluation system on mobile device 50. In some embodiments, the application(s) corresponding to the retinopathy evaluation system may be directly launched on the mobile device 50 as an app.

If this is the first time a user is accessing the retinopathy evaluation system, then the user may be prompting to enter his/her login information. In some embodiments, the user may have to create a login. In this case, the user may have to input their name and other identifying information, such as biometric information (e.g., information that could be obtained using biometric measurements), which may be used to grant the user access to the application. In some embodiments, only pre-authorized users may access the application, in which case the user may have received a username previously and he can use that to log into the application. The first time a user accesses the retinopathy evaluation system, the mobile device 50 may need network access to perform login validation in some embodiments.

Any subsequent time the user accesses the retinopathy evaluation system, the mobile device 50 may not require network access to validate login. In this case, the mobile device 50 may have stored the login information, and can allow access to the user by locally validating the login information. In some embodiments, the user may not need to enter his or her login information any subsequent time he or she accesses the system. In other embodiments, the mobile device 50 may request additional information for verification purposes, such as an answer to a security question. In yet another embodiment, the mobile device 50 may require network access to allow user to access the application. Alternatively, the mobile device 50 may only request additional verification information if the mobile device 50 is not connected to a network.

Once the user accesses the retinopathy evaluation system, the graphical user interface module 210, at block 302, displays a user interface on mobile device 50. At this time, the user may be prompted to select an image to view and/or evaluate. The graphical user interface module 210 can display a gallery of images from which the user can select an image to view. FIG. 5 is an example user interface screen 402 illustrating a gallery of images from which the user can select one or more to view. In some embodiments, the gallery may contain thumbnails of the images, as shown in screen 402 of FIG. 5. In some embodiments, the gallery may be displayed in a separate window on top of an application home-screen. The home-screen may be the initial screen that is displayed as part of the retinopathy workflow, evaluation and grading application.

In some embodiments, general information, such as right eye, left eye, patient identification information, and the like, may also be displayed with the images. The user can select an image by using his or her finger and tapping the touch-screen display. In an alternative embodiment that also employs a pointing device, such as a mouse, the user can use a mouse to select an image. The images may be preloaded on mobile device 50, or the user may use other tools to obtain the images. For example, the user may download images using network 105 or transfer images from an external memory drive. In some embodiments, the user may be able to take an image of a retina using an appropriate imaging device.

Once the user selects an image, the graphical user interface module 210, at block 302, provides a user interface on the mobile device 50 that displays the selected retina image, and a first plurality of user input controls representing categories of features related to retinopathy evaluation. In an example embodiment, the categories of features include HMA (hemorrhages and microaneurysms), VB (venous beading), IRMA (intraretinal microvascular abnormalities), HE (hard exudates), NVD (new vessels on the disc), NVE (new vessels elsewhere), VH (vitreous hemorrhage), and PVH (preretinal vitreous hemorrhage), and NPDR (nonproliferative diabetic retinopathy). In some embodiments, a button corresponding to a group of categories is displayed. For example, PDR (proliferative diabetic retinopathy) is a group of categories including NVD, NVE, VH, and PVH, and NPDR (nonproliferative diabetic retinopathy) is a group of categories including HMA, VB, IRMA, and HE.

The graphical user interface module 210 may display example screen 404 shown in FIG. 6. Screen 404 includes buttons 505 (HMA), 510 (VB), 515 (IRMA), 520 (HE), 525 (PDR), button 610 (Gallery), and image 710. Screen 404 displays the selected image in the center of the screen, as shown by image 710. The buttons 505, 510, 515, 520, 525 and button 610 are displayed on the edge of the screen so that they do not block the user's view of image 710. Screen 404 shows the user input controls, representing categories of features, as buttons 505, 510, 515, 520, and a group of features as button 525. The categories of features are discussed in detail below. Screen 404 also includes button 610 labeled “Gallery.” The user can select button 610 to display the gallery of images, as shown by screen 402 of FIG. 5. As discussed above, the user can select an image from the gallery to view. Even though screen 404 displays buttons 505, 510, 515, 520, 525, button 610, and image 710 in a particular order and at a particular place in screen 404, it should be understood that in other embodiments these elements may be displayed in a different potion of the user interface screen and/or in a different order.

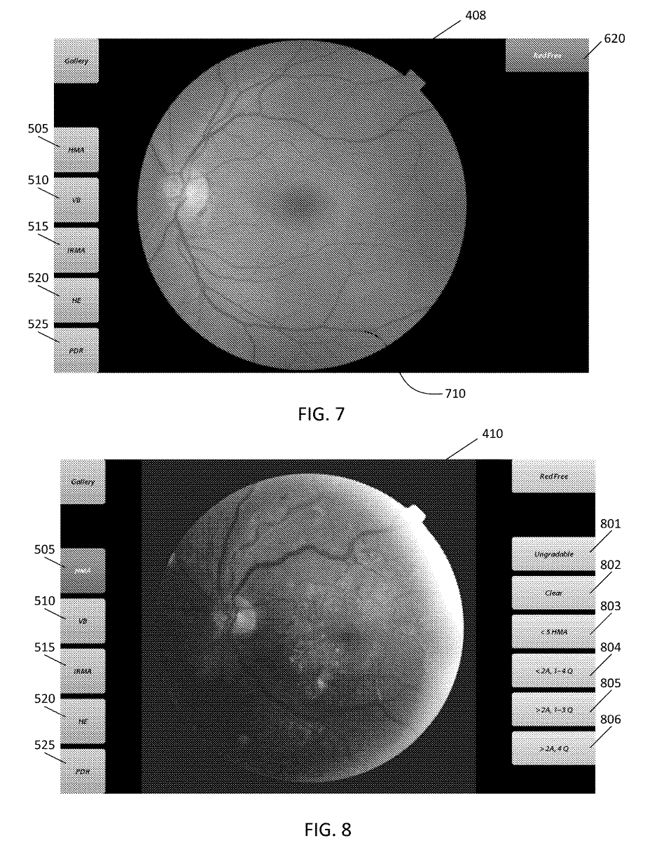

In some embodiments, the user can modify the display of the retina image by removing the red color information or channel, which improves the contrast of vessels and other structures. In an example embodiment, the user can select button 620 shown in FIG. 7 to view a red-free image. FIG. 7 shows an example screen 408 displaying the selected retina image 710 as a red-free image. For illustrative purposes, the remaining figures of example user interfaces use an image of a retina showing significant retinal damage.

At block 304, the user input module 220 receives a selection of a category of feature via the user interface displayed on mobile device 50. For example, the user may select button 505 representing the category of features called hemorrhages and microaneurysms (HMA), as shown in screen 410 of FIG. 8. As seen in FIG. 8, button 505 is highlighted to indicate that it is presently selected or active.

At block 306, the graphical user interface module 210 displays a second plurality of user input controls corresponding to a set of features that are associated with the selected category of features, in response to receiving the selection of the category. Continuing with the example where the user selects button 505 representing the HMA category, the graphical user interface module 210 displays the set of features as shown by buttons 801-806 in screen 410 of FIG. 8. In an example embodiment, the HMA category, according to the ETDRS grading protocols that compare assessed lesion extent according to number of Quadrants (Q) involved in ETDRS standard photograph 2A, includes the following six features: Ungradable; Clear; <5 HMA; <2A, 1-4Q; >2A, 1-3Q; and >2A, 4Q. Even though screen 410 shows particular elements, such as buttons 505, 510, 515, 520, 525 and 801-806 in particular areas, it should be understood that in alternative embodiments the elements may be displayed in a different portion of the user interface screen, in a different order, and/or may be labeled differently.

The Ungradable feature represents the user's observation that the retina image cannot be graded for hemorrhages or microaneurysms by the user based on viewing the retina image. The Clear feature represents the user's observation that the retina image is clear of any hemorrhages or microaneurysms. The <5HMA feature represents the user's observation that the retina image consists of less than five hemorrhages or microaneurysms. The <2A, 1-4Q feature represents the user's observation that the retina image consists of hemorrhages or microaneurysms less than that of standard image 2A in one to four quadrants. Standard image 2A is a commonly used reference image associated with the ETDRS classification system. The >2A, 1-3Q feature represents the user's observation that the retina image contains hemorrhages or microaneurysms more than standard image 2A in one to three quadrants. The >2A, 4Q feature represents the user's observation that the retina image has hemorrhages or microaneurysms more than standard image 2A in four quadrants.

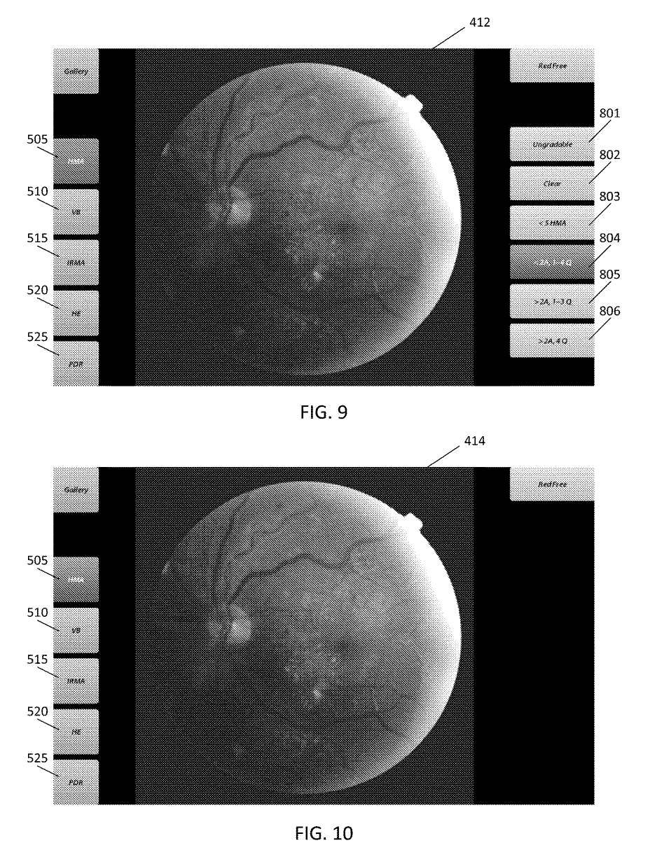

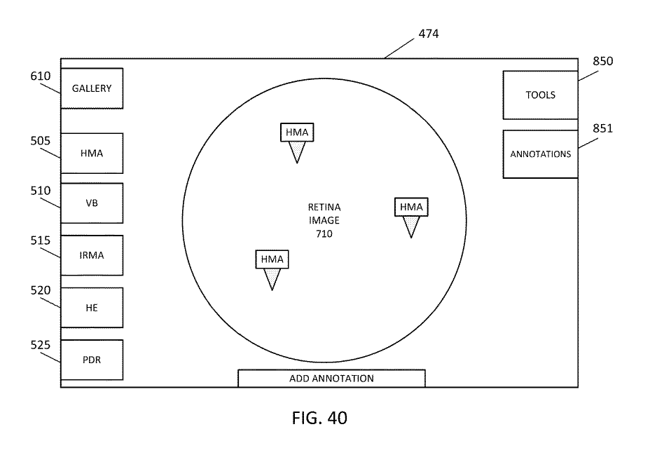

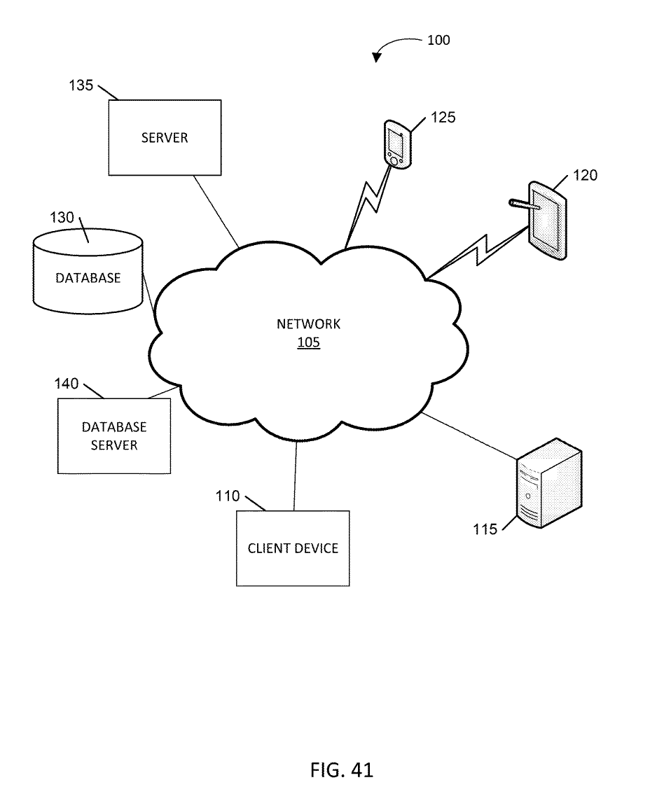

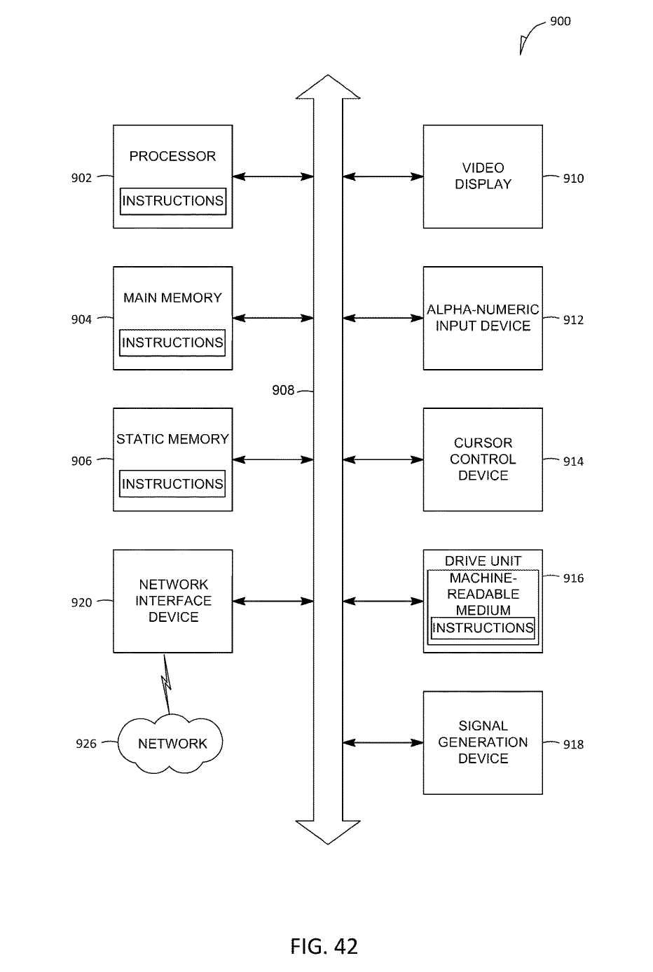

At block 308, the user input module 220 receives a selection of a feature from the set of displayed features via the user interface. For example, the user may select button 804 representing the feature called ‘<2A, 1-4Q,’ as shown in screen 412 of FIG. 9. As shown in FIG. 9, button 804 is highlighted by a color change indicating that the button is selected or active. This may be described as the active category color change. Once the selection of the feature is received, the graphical user interface module 210 displays screen 414 of FIG. 10, in which the buttons representing the features are not displayed. Button 505 representing the HMA category of features is highlighted in a different color than FIGS. 8-9 to indicate that the user has entered information or that information has been saved for this category (e.g., the selection of a feature from the set of features associated with the category). This may be described as the completed category color change. In some embodiments, tools are provided for marking retinal features such as hemorrhages or microaneurysms. Such markings can be made before or after selection of the appropriate feature button. Further details regarding marking tools are provided below with respect to the description of FIG. 40. FIG. 10 is an example screen 414 showing that information in the form of a selection of feature <2A, 1-4Q has been received for the HMA category by the color of button 505.

At block 310, the retinopathy workflow module 230 determines whether enough information has been provided by the user to produce a diagnosis or treatment recommendation. If more information is required, the method 300 jumps to step 304 and continues through blocks 306 and 308 for an additional category and associated set of features. If enough information is provided, the method 300 continues to block 312.

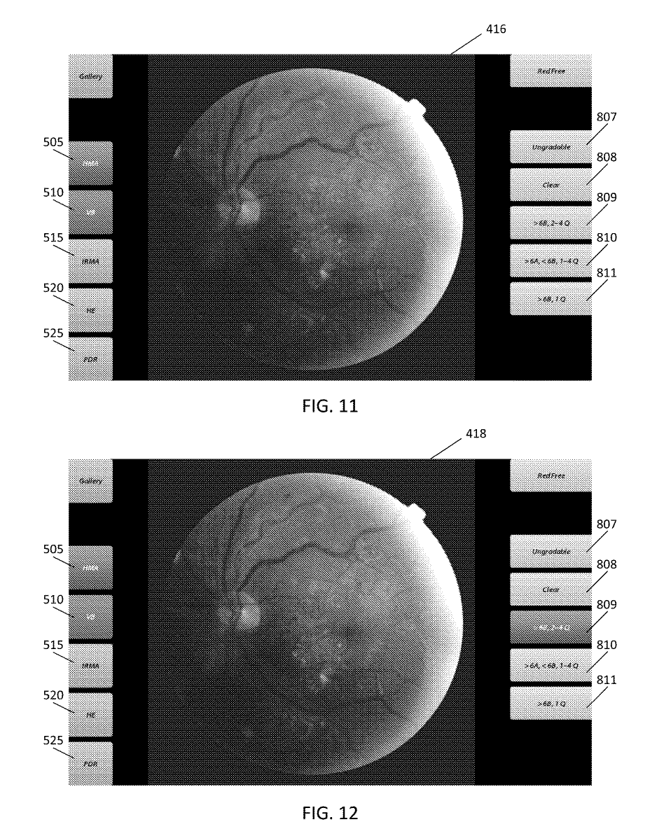

Continuing with the previous example, enough information has not been provided. FIG. 10 shows button 505 highlighted in green color as a result of the completed category persistent color change, while the other buttons 510, 515, and 520 are not highlighted; thereby indicating to the user that information needs to be entered for categories corresponding to buttons 510, 515, and 520. In this example, at block 304 the user input module 220 receive a selection of a second category of features. FIG. 11 is an example screen 416 where the user selects the venous beading (VB) category of features by selecting button 510, and in response to the selection, the graphical interface module 210 displays the corresponding features on the user interface (e.g., screen 416) per block 306 via buttons 807-811. Button 510 is highlighted to indicate that it is selected or active. As shown in FIG. 11, screen 416 displays buttons 807-811 as the features corresponding the selected VB category. In an example embodiment, the VB category has the following five features associated with it: Ungradable; Clear; >6B, 2-4Q; >6A, <6B, 1-4Q; and >6B, 1Q. Even though screen 416 shows particular elements, such as buttons 505, 510, 515, 520, 525 and 807-811, it should be understood that in alternative embodiments the elements may be displayed in a different portion of the user interface screen, in a different order, and/or may be labeled differently.

The Ungradable feature represents the user's observation that venous beading cannot be graded by the user based on viewing the retina image. The Clear feature represents the user's observation that the retina image is clear of any venous beading. The >6B, 2-4Q feature represents the user's observation that the retina image consists of venous beading greater than standard image 6B in two to four quadrants. The >6A, <6B, 1-4Q feature represents the user's observation that the retina image consists of venous beading greater than standard image 6A, but less than standard image 6B in one to four quadrants. Standard image 6A and standard image 6B are commonly used reference images associated with the ETDRS classification system. The >6B, 1Q feature represents the user's observation that the retina image consists of venous beading greater than standard image 6B in one quadrant.

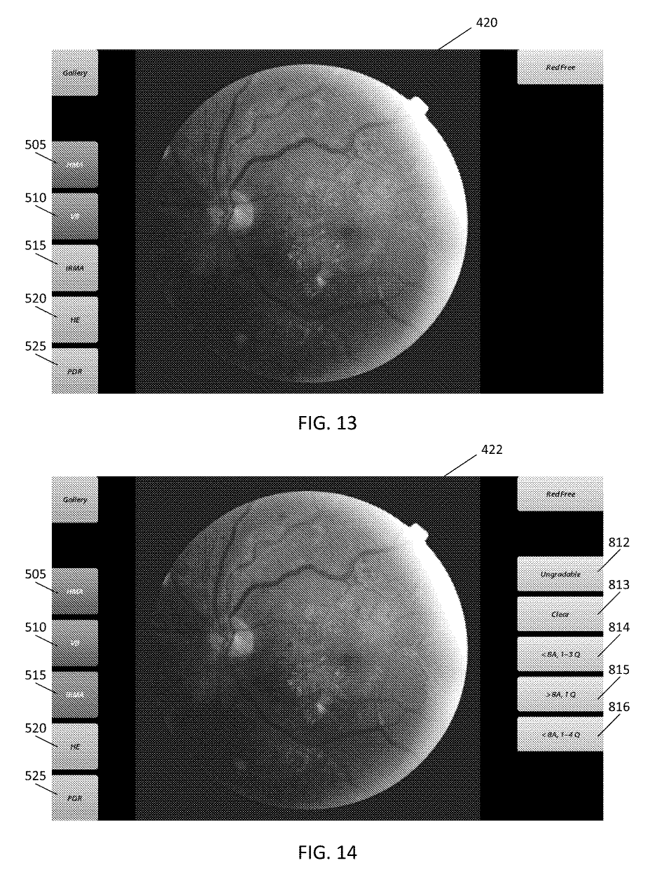

At block 308, the user input module 220 receives a selection of a feature from the set of displayed features via the user interface. For example, the user may select button 809 representing the feature called ‘>6B, 2-4Q,’ as shown in screen 418 of FIG. 12. As shown in FIG. 12, button 809 is highlighted indicating that the button is selected or active. FIG. 12 is an example screen 418 showing the feature selected by the user for the VB category of features. Once the selection of the feature is received, the graphical user interface module 210 displays screen 420 of FIG. 13, in which the buttons representing the features are not displayed. Button 510 representing the VB category of features is highlighted in a different color than that of buttons 515 and 520, which correspond to categories that have not yet been selected for this retina image, to indicate that the user has entered information or that information has been saved for the VB category. In some embodiments, tools are provided for marking retinal features such as venous beading. Such markings can be made before or after selection of the appropriate feature button. Further details regarding marking tools are provided below with respect to the description of FIG. 40. FIG. 13 is an example screen 420 showing that information has been received for the VB category.

Continuing with the example, the retinopathy workflow module 230 determines that enough information has not been provided per block 310. FIG. 13 shows buttons 505 and 510 highlighted in green color, while the buttons 515, and 520 are not highlighted, thereby indicating to the user that information needs to be entered for categories corresponding to buttons 515 and 520. In this example, at block 304 the user input module 220 receives a selection of a third category of features. FIG. 14 is an example screen 422 where the user selects the intraretinal microvascular abnormalities (IRMA) category of features by selecting button 515, and in response to the selection, the corresponding features are displayed via buttons 812-816 per block 306. Button 515 is highlighted to indicate that it is selected or active. As shown in FIG. 14, screen 422 displays buttons 812-816 as the features corresponding to the selected IRMA category. In an example embodiment, the IRMA category has the following five features associated with it: Ungradable; Clear; <8A, 1-3Q; >8A, 1Q; and <8A, 1-4Q. Even though screen 422 shows particular elements, such as buttons 505, 510, 515, 520, 525 and 812-816, it should be understood that in alternative embodiments the elements may be displayed in a different portion of the user interface screen, in a different order, and/or may be labeled differently.

The Ungradable feature represents the user's observation that grading of any intraretinal microvascular abnormalities cannot be determined by the user based on viewing the retina image. The Clear feature represents the user's observation that the retina image is clear of any intraretinal microvascular abnormalities. The <8A, 1-3Q feature represents the user's observation that the retina image consists of intraretinal microvascular abnormalities less than standard image 8A in one to three quadrants. Standard image 8A is a commonly used reference image associated with the ETDRS classification system. The >8A, 1Q feature represents the user's observation that the retina image consists of intraretinal microvascular abnormalities greater than standard image 8A in one quadrant. The <8A, 1-4Q feature represents the user's observation that the retina image consists of intraretinal microvascular abnormalities greater than standard image 8A in one to four quadrants.

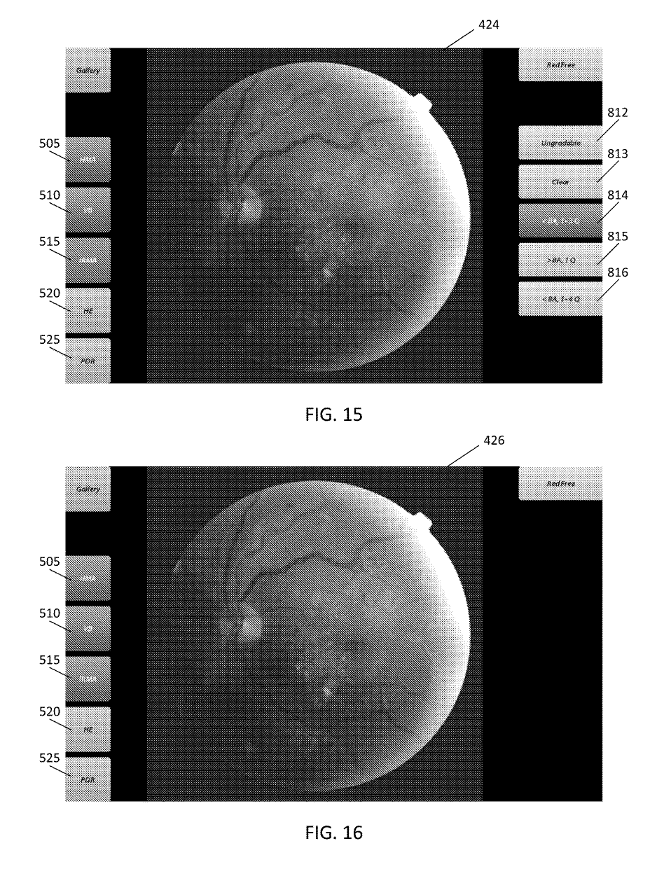

At block 308, the user input module 220 receives a selection of a feature from the set of displayed features via the user interface. For example, the user may select button 814 representing the feature called ‘<8A, 1-3Q,’ as shown in screen 424 of FIG. 15. As shown in FIG. 15, button 814 is highlighted indicating that the button is selected or active. FIG. 15 is an example screen 424 showing the feature selected by the user for the IRMA category of features. Once the selection of the feature is received, the graphical user interface module 210 displays screen 426 of FIG. 16, in which the buttons representing the features are not displayed. Button 515 representing the IRMA category of features is highlighted in a different color than that of button 520 to indicate that the user has entered information or that information has been saved for this category. In some embodiments, tools are provided for marking retinal features such as intraretinal microvascular abnormalities. Such markings can be made before or after selection of the appropriate feature button. Further details regarding marking tools are provided below with respect to the description of FIG. 40. FIG. 16 is an example screen 426 showing that information has been received for the IRMA category.

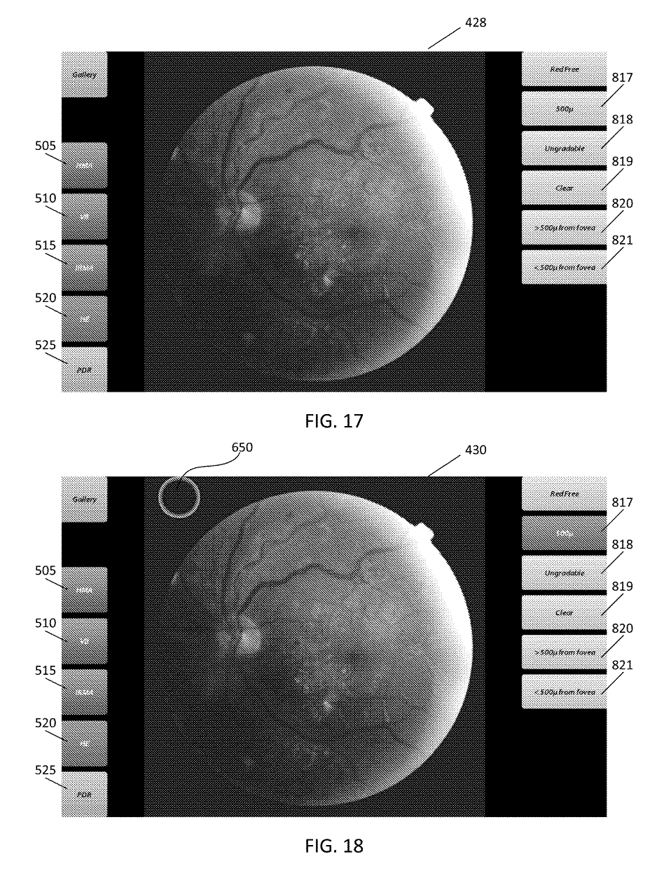

Continuing with the example, the retinopathy workflow module 230 determines that enough information has not been provided per block 310. FIG. 16 shows buttons 505, 510 and 515 highlighted in green color, while the button 520 is not highlighted; thereby indicating to the user that information needs to be provided for the category corresponding to button 520. In this example, at block 304 the user input module 220 receive a selection of a fourth category of features. FIG. 17 is an example screen 428 where the user selects the hard exudates (HE) category of features by selecting button 520, and in response to the selection, the corresponding features are displayed via buttons 817-821. Button 520 is highlighted to indicate that it is selected or active. At block 306, the graphical user interface module 210 displays the corresponding features on the user interface in response to the selection of the selected fourth category. As shown in FIG. 17, screen 428 displays buttons 817-821 as the features corresponding to the selected HE category. In an example embodiment, the HE category has the following five features associated with it: 500μ; Ungradable; Clear; >500μ from fovea; and <500μ from fovea. Even though screen 422 shows particular elements, such as buttons 505, 510, 515, 520, 525 and 812-816, it should be understood that in alternative embodiments the elements may be displayed in a different portion of the user interface screen, in a different order, and/or may be labeled differently.

In an example embodiment, selection of the 500μ feature displays an annular, circular measurement aid (discussed below). This ring has a radius of 500 microns (μ) of the retina image. In some embodiments, other numerical values may be used for the radius of the ring, for examples, 1,500μ, depending upon local clinical workflow practice. The user can position the ring, centered over the fovea, for use as a guide in determining whether hard exudates are present within 500μ from the fovea or lie over 500μ from the fovea. The Ungradable feature represents the user's observation that the presence and position of any hard exudates cannot be determined by the user based on viewing the retina image. The Clear feature represents the user's observation that the retina image is clear of any hard exudates. The >500μ from fovea feature represents the user's observation that the retina image has hard exudates more than 500μ from the center of the fovea. The <500μ from fovea feature represents the user's observation that the retina image shows hard exudates within 500μ of the center of the fovea.

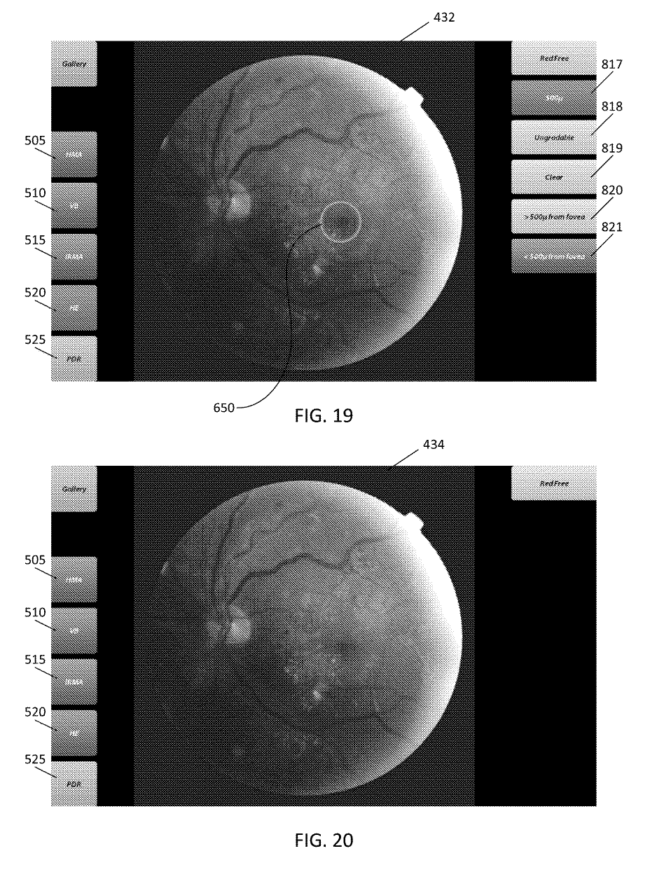

At block 308, the user input module 220 receives a selection of a feature from the set of displayed features via the user interface. FIG. 18 is an example screen 430 showing the feature selected by the user for the HE category of features. The user may select button 817 representing the feature called 500μ as shown in screen 430 of FIG. 18. As shown in FIG. 18, button 817 is highlighted indicating that the button is selected or active. In some embodiments, selecting button 817 displays a marker like circle 650. The user can select the marker and move it, so he can indicate where the fovea is in the retina image, and also mark a 500μ region around the fovea, as shown in FIG. 19 by circle 650. The circle 650 also zooms and pans along with the retina image. After marking the fovea, the user can select any of buttons 819-821 that represents his/her observation. In this example, the user selects feature ‘<500μ from fovea’ via button 821. As shown in FIG. 19, button 821 is highlighted indicating that the button is selected or active. Once the selection of the feature is received, the graphical user interface module 210 displays screen 434 of FIG. 20, in which the buttons representing the features are not displayed. Button 520 representing the HE category of features is highlighted in a different color than shown in FIGS. 17-19 to indicate that the user has entered information or that information has been saved for this category. In some embodiments, tools are provided for marking retinal features such as hard exudates. Such markings can be made before or after selection of the appropriate feature button. Further details regarding marking tools are provided below with respect to the description of FIG. 40. FIG. 20 is an example screen 434 showing that information has been received for the HE category.

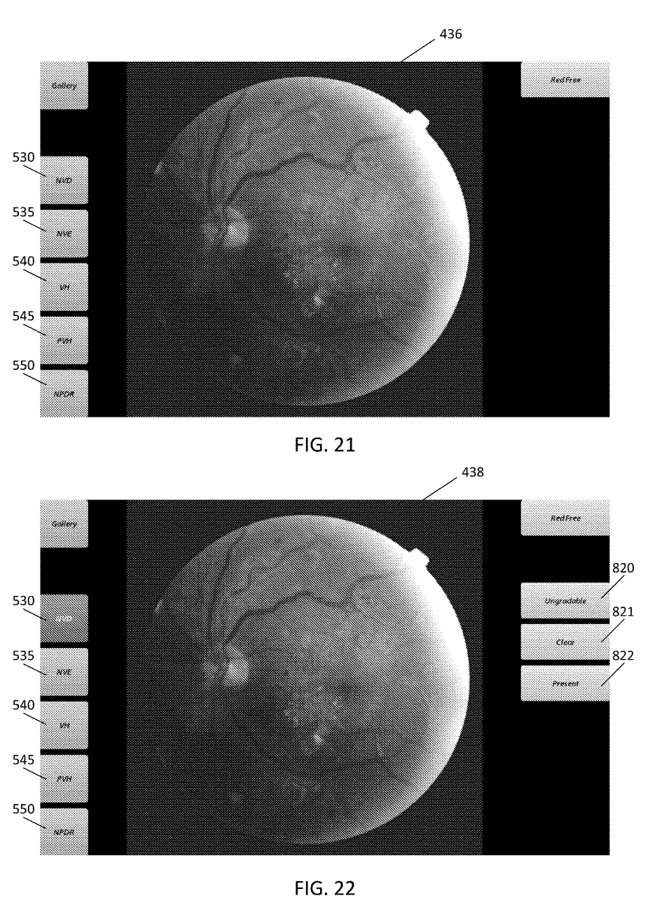

Continuing with the example, the retinopathy workflow module 230 determines that enough information has not been provided per block 310. FIG. 20 shows buttons 505-520 highlighted in green color, indicating that information has been entered for categories corresponding to buttons 505, 510, 515, and 520. In some embodiments, not all buttons corresponding to categories are displayed together. For example, buttons corresponding to categories may be grouped. Button 525 corresponds to a group of categories labeled proliferative diabetic retinopathy (PDR). In this embodiment, selecting button 525 corresponding to PDR displays another list of categories of features as shown by buttons 530, 535, 540, and 545 in screen 436 of FIG. 21 in place of the buttons 505, 510, 515, and 520. Thus, the PDR button 525 leads the user to a new group of categories of features that is related to PDR. In some embodiments, button 525 (PDR) toggles to button 550 (NPDR) as shown in screen 436 of FIG. 21. In this manner, the last buttons (525 and 550) on the left side of the screen toggle to display a set of categories of features related to either PDR or NPDR. Selecting the NPDR button 550 returns the user to previous group of categories (HMA, VB, IRMA, and HE), as discussed below.

Selection of button 525 on screen 434 (shown in FIG. 20) displays buttons 530, 535, 540, and 545, which are not highlighted (as shown in FIG. 21), indicating to the user that information needs to be entered for categories NVD, NVD, VH, and PVH. FIG. 22 is an example screen 438 where the user selects the new vessels on the optic disc (NVD) category of features by selecting button 530, and in response to the selection, the corresponding features are displayed via buttons 820-822. Button 530 is highlighted to indicate that it is selected or active. At block 306, the graphical user interface module 210 displays the corresponding features on the user interface in response to the selection of the selected fifth category. As shown in FIG. 22, screen 438 displays buttons 820-822 as the features corresponding to the selected NVD category. In an example embodiment, the NVD category has the following three features associated with it: Ungradable, Clear, and Present. Even though screen 438 shows particular elements, such as buttons 530, 535, 540, and 545 and 820-822, it should be understood that in alternative embodiments the elements may be displayed in a different portion of the user interface screen, in a different order, and/or may be labeled differently. The Ungradable feature represents the user's observation that the presence or absence of any new vessels on the disc cannot be determined by the user based on viewing the retina image. The Clear feature represents the user's observation that the retina image is clear of any new vessels on the disc. The Present feature the user's observation that the retina image shows new vessels on the disc.

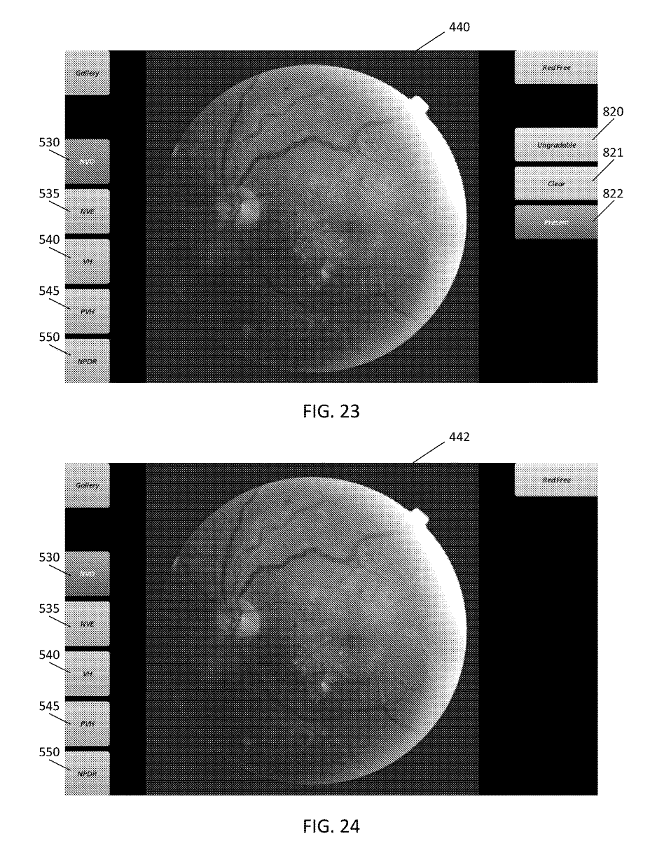

At block 308, the user input module 220 receives a selection of a feature from the set of displayed features via the user interface. For example, the user may select button 822 representing the feature called ‘Present’ as shown in screen 440 of FIG. 23. As shown in FIG. 23, button 822 is highlighted indicating that the button is selected or active. FIG. 23 is an example screen 440 showing the feature selected by the user for the NVD category of features. Once the selection of the feature is received, the graphical user interface module 210 displays screen 442 of FIG. 24, in which the buttons representing the features are not displayed. Button 530 representing the NVD category of features is highlighted in a different color than that of buttons 535, 540, and 545 to indicate that the user has entered information or that information has been saved for this category corresponding to button 530. In some embodiments, tools are provided for marking retinal features such as new vessels on the optic disc. Such markings can be made before or after selection of the appropriate feature button. Further details regarding marking tools are provided below with respect to the description of FIG. 40. FIG. 24 is an example screen 442 showing that information has been received for the NVD category.

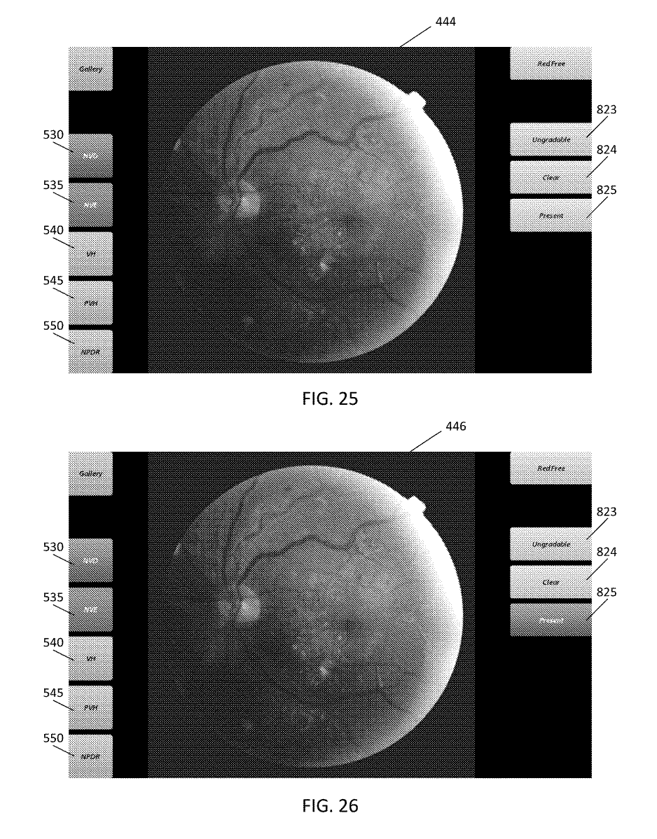

Further, continuing with the example, the retinopathy workflow module 230 determines that enough information has not been provided per block 310. FIG. 24 shows button 530 highlighted in green color, while the buttons 535, 540, 545 are not highlighted; thereby indicating to the user that information needs to be entered for categories corresponding to buttons 535, 540, and 545. In this example, at block 304 the user input module 220 receives a selection of a sixth category of features. FIG. 25 is an example screen 444 where the user selects the new vessels elsewhere (NVE) category of features by selecting button 535, and in response to the selection, the corresponding features are displayed via buttons 823-825. Button 535 is highlighted to indicate that it is selected or active. At block 306, the graphical user interface module 210 displays the corresponding features on the user interface in response to the selection of the selected sixth category. As shown in FIG. 25, screen 444 displays buttons 823-825 as the features corresponding to the selected NVE category. In an example embodiment, the NVE category has the following three features associated with it: Ungradable, Clear, and Present. Even though screen 444 shows particular elements, such as buttons 530, 535, 540, and 545 and 823-825, it should be understood that in alternative embodiments the elements may be displayed in a different portion of the user interface screen, in a different order, and/or may be labeled differently. The Ungradable feature represents the user's observation that the presence or absence of new vessels, other than any on the optic disc, cannot be determined based on viewing the retina image. The Clear feature represents the user's observation that the retina image is clear of any new vessels elsewhere. The Present feature indicated the user's observation that the retina image consists of new vessels in an area other than the disc.

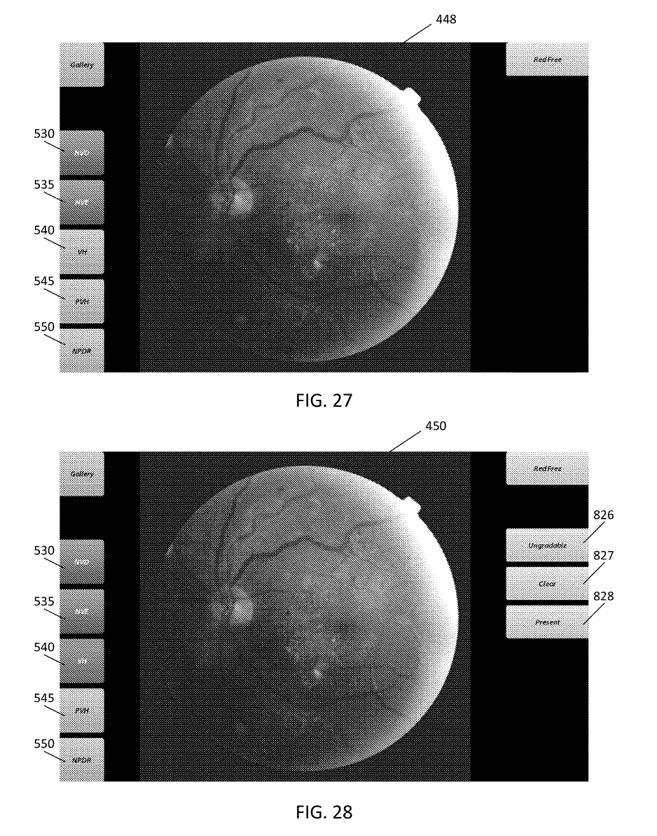

At block 308, the user input module 220 receives a selection of a feature from the set of displayed features via the user interface. For example, the user may select button 825 representing the feature called ‘Present’ as shown in screen 446 of FIG. 26. As shown in FIG. 26, button 825 is highlighted indicating that the button is selected or active. FIG. 26 is an example screen 446 showing the feature selected by the user for the NVE category of features. Once the selection of the feature is received, the graphical user interface module 210 displays screen 448 of FIG. 27, in which the buttons representing the features are not displayed. Button 535 representing the NVE category of features is highlighted in a different color than FIGS. 25-26 to indicate that the user has entered information or that information has been saved for this category. In some embodiments, tools are provided for marking retinal features such as new vessels elsewhere. Such markings can be made before or after selection of the appropriate feature button. Further details regarding marking tools are provided below with respect to the description of FIG. 40. FIG. 27 is an example screen 448 showing that information has been received for the NVE category.

Further, continuing with the example, the retinopathy workflow module 230 determines that enough information has not been provided per block 310. FIG. 27 shows buttons 530 and 535 highlighted in green color, while the buttons 540 and 545 are not highlighted; thereby indicating to the user that information needs to be entered for categories corresponding to buttons 540 and 545. In this example, at block 304 the user input module 220 receives a selection of a seventh category of features. FIG. 28 is an example screen 450 where the user selects the vitreous hemorrhage (VH) category of features by selecting button 540, and in response to the selection, the corresponding features are displayed via buttons 826-828. Button 540 is highlighted to indicate that it is selected or active. At block 306, the graphical user interface module 210 displays the corresponding features on the user interface in response to the selection of the selected seventh category. As shown in FIG. 28, screen 450 displays buttons 826-828 as the features corresponding to the selected VH category. In an example embodiment, the VH category has the following three features associated with it: Ungradable, Clear, and Present. Even though screen 450 shows particular elements, such as buttons 530, 535, 540, and 545 and 826-828, it should be understood that in alternative embodiments the elements may be displayed in a different portion of the user interface screen, in a different order, and/or may be labeled differently. The Ungradable feature represents the user's observation that the presence or absence of vitreous hemorrhage cannot be determined by the user based on viewing the retina image. The Clear feature represents the user's observation that the retina image is clear of any vitreous hemorrhage. The Present feature the user's observation that the retina image shows the presence of vitreous hemorrhage.

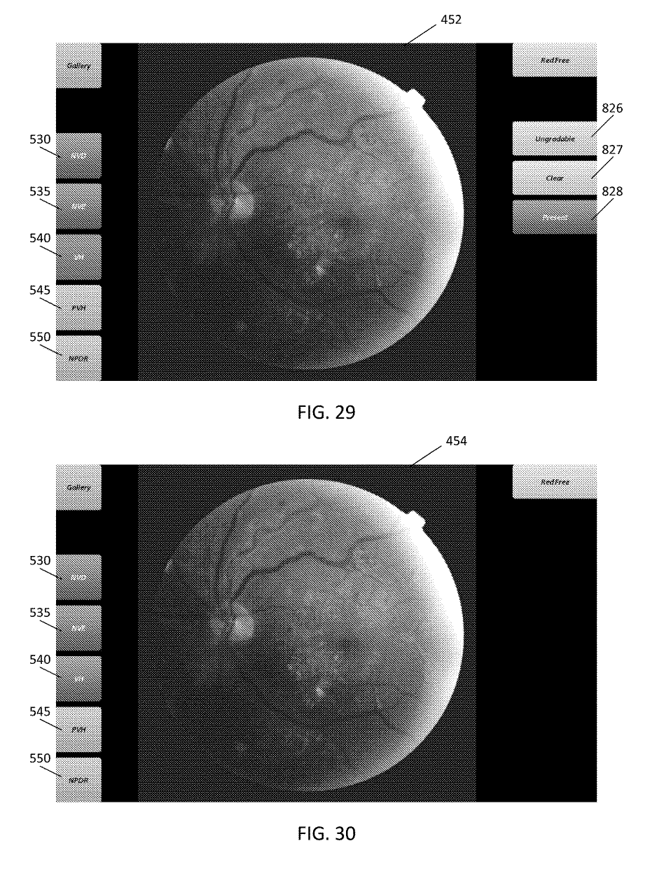

At block 308, the user input module 220 receives a selection of a feature from the set of displayed features via the user interface. For example, the user may select button 828 representing the feature called ‘Present’ as shown in screen 452 of FIG. 29. As shown in FIG. 29, button 828 is highlighted indicating that the button is selected or active. FIG. 29 is an example screen 452 showing the feature selected by the user for the VH category of features. Once the selection of the feature is received, the graphical user interface module 210 displays screen 454 of FIG. 30, in which the buttons representing the features are not displayed. Button 540 representing the VH category of features is highlighted in a different color than FIGS. 28-29 to indicate that the user has entered information or that information has been saved for this category. In some embodiments, tools are provided for marking retinal features such as vitreous hemorrhage. Such markings can be made before or after selection of the appropriate feature button. Further details regarding marking tools are provided below with respect to the description of FIG. 40. FIG. 30 is an example screen 454 showing that information has been received for the VH category.

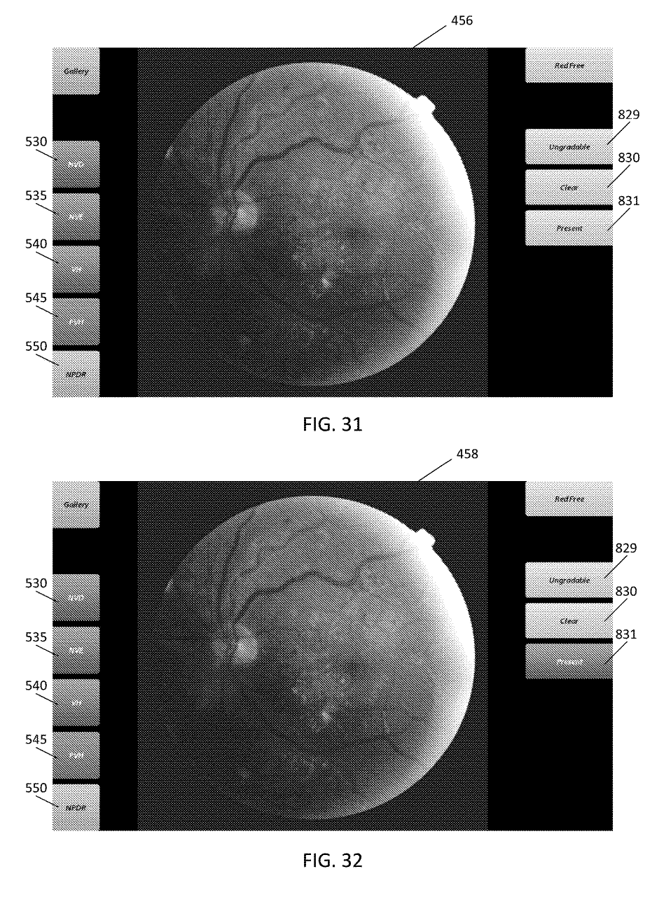

Continuing with the example, the retinopathy workflow module 230 determines that enough information has not been provided. FIG. 30 shows buttons 530, 535, 540 highlighted in green color, while the button 545 is not highlighted; thereby indicating to the user that information needs to be entered for the category corresponding to button 545. In this example, at block 304 the user input module 220 receive a selection of a eighth category of features. FIG. 31 is an example screen 456 where the user selects the preretinal vitreous hemorrhage (PVH) category of features by selecting button 545, and in response to the selection, the corresponding features are displayed via buttons 829-831. Button 545 is highlighted to indicate that it is selected or active. At block 306, the graphical user interface module 210 displays the corresponding features on the user interface in response to the selection of the selected eighth category. As shown in FIG. 31, screen 456 displays buttons 829-831 as the features corresponding to the selected PVH category. In an example embodiment, the PVH category has the following three features associated with it: Ungradable, Clear, and Present. Even though screen 456 shows particular elements, such as buttons 530, 535, 540, and 545 and 829-831, it should be understood that in alternative embodiments the elements may be displayed in a different portion of the user interface screen, in a different order, and/or may be labeled differently. The Ungradable feature represents the user's observation that the presence or absence of preretinal vitreous hemorrhage cannot be determined by the user based on viewing the retina image. The Clear feature represents the user's observation that the retina image is clear of any preretinal vitreous hemorrhage. The Present feature the user's observation that the retina image shows preretinal vitreous hemorrhage.

At block 308, the user input module 220 receives a selection of a feature from the set of displayed features via the user interface. For example, the user may select button 831 representing the feature called ‘Present’ as shown in screen 458 of FIG. 32. As shown in FIG. 32, button 831 is highlighted indicating that the button is selected or active. FIG. 32 is an example screen 458 showing the feature selected by the user for the PVH category of features. Once the selection of the feature is received, the graphical user interface module 210 displays screen 460 of FIG. 33, in which the buttons representing the features are not displayed. Button 545 representing the PVH category of features is highlighted in a different color than FIGS. 31-32 to indicate that the user has entered information or that information has been saved for this category. In some embodiments, tools are provided for marking retinal features such as preretinal vitreous hemorrhage. Such markings can be made before or after selection of the appropriate feature button. Further details regarding marking tools are provided below with respect to the description of FIG. 40. FIG. 33 is an example screen 460 showing that information has been received for the PVH category.

Even though the example user interface screens shown in FIGS. 6-33 and 38-40, display buttons arranged in a vertical column on each side of the user interface screen, it should be understood that the buttons may be arranged along an arc on each side of the user interface screen. Providing the buttons along an arc in the user interface screen leverages the arc-type motion of a user's thumbs and fingers. For example, as discussed in relation to FIG. 4, the buttons may be provided in the areas 604a and 604b, along arc 603a and 603b so that they are within the range of motion of the user's thumbs (see 606a and 606b of FIG. 4).

At this point the user has entered information for each of the retinal lesion categories of features necessary for determining a diagnosis and management/treatment recommendation. At block 310, the retinopathy workflow module 230 determines that enough information has been provided by the user to produce diagnosis information or treatment recommendation, and method 300 proceeds to block 312. Furthermore, button 630 is displayed in screen 460 of FIG. 33 to indicate to the user that he or she can submit the information to determine a diagnosis.

Note that button 550 is not highlighted. Selecting button 550 at any time, brings up the previous list of categories (buttons 505, 510, 515, and 520) and button 525 for the PDR group of categories. If the user selects button 550 at this point, then the user interface will display buttons 505, 510, 515, and 520 highlighted in green to indicate that the categories have information associated with them. Additionally, PDR button 525 will be displayed with no highlighting. Selecting PDR button 525 will return the user to screen 460 with buttons 530-545 highlighted. In this manner, the last button in the list of categories of features can be toggled to switch between the PDR and NPDR categories. This mechanism also reduces the number of buttons displayed on the user interface at the same time, so that the user is not overwhelmed with numerous data entry fields like conventional form-entry systems for retinopathy evaluation. Additionally, this mechanism also leaves sufficient space on a single mobile-device sized screen for displaying the retina image.

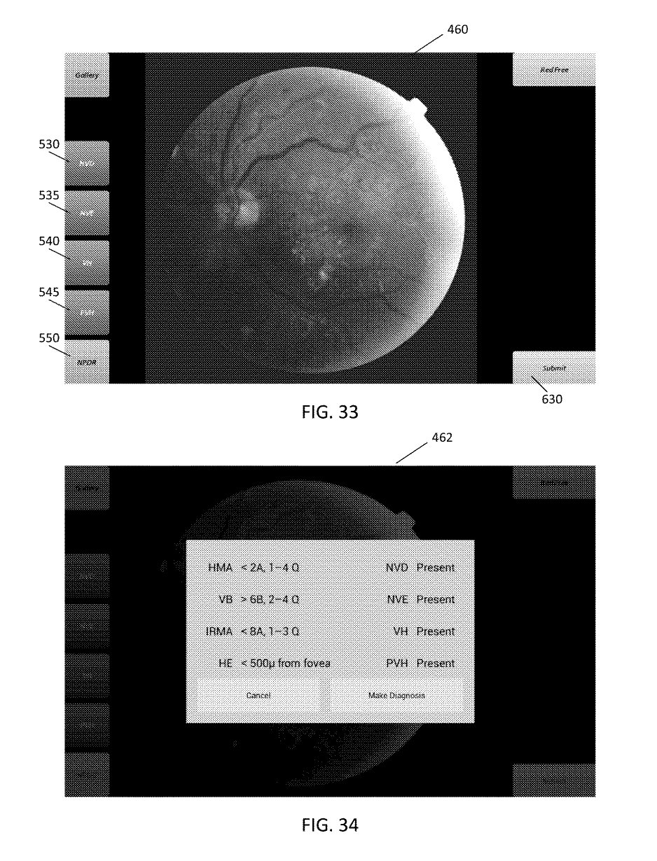

Referring back to the example, the user can select submit button 630 on screen 460 of FIG. 33. Selection of button 630 displays a dialog box, in an example embodiment, that summarizes the information entered by the user, as shown in screen 462 of FIG. 34. If this information appears to be accurate, the user then can select the ‘Make Diagnosis’ button; otherwise the user can select the ‘Cancel’ button to return to the user input screens to review or revise the information entered. A user may continue to make changes and revise his or her selections until the selections and findings are saved. When the findings are saved, a record is created and saved, at which point the user cannot edit the saved record. In some embodiments, the user or another user may view a previously saved evaluation/record. Making changes to a previously saved evaluation creates a new record (as an addendum or amendment to the previous evaluation/previous record).

If the user selects the ‘Make Diagnosis’ button, at block 312, the treatment recommendation module 240 determines information related to a diagnosis and a management/treatment recommendation based on the inputs provided by the user. Information related to a diagnosis may include no evidence of retinopathy, mild NPDR, moderate NPDR, severe NPDR, PDR, no diabetic macular edema, diabetic macular edema, clinically significant macular edema, or no diagnosis/ungradable. Information related to treatment recommendation may include a recommendation for reevaluation and/or a recommendation for a referral (e.g, reevaluate in 1 year, reevaluate in 6 months, refer to ophthalmologist within 4 months, refer to ophthalmologist within 2 months, refer to ophthalmologist within 6 months, and refer for dilated eye examination). Information related to management recommendation may include managing hypertension, lipids or blood glucose levels, taking medications as prescribed by doctor, and recommendations around healthy lifestyles including eating more fruits and vegetables, quitting smoking, consuming less alcohol, and increasing physical activity. The treatment recommendation module 240 can determine the appropriate diagnosis information and management/treatment recommendation based on an example workflow (discussed below in detail) included in the retinopathy evaluation system. When the user selects the ‘Make Diagnosis’ button, a record is created and stored. The selected features, diagnosis information and management/treatment recommendations are also stored as part of the record. The record also includes user identification information and/or a digital signature associated with the user. The record can also include a date and time stamp indicating when the evaluation was made or saved. The user can select the ‘OK’ button to return to the retinopathy evaluation system.

The example workflow aids in determining a diagnosis by associating each selected feature to an image finding, based on professional medical standards and ETDRS, and then assigning a diagnosis identification (ID) number and a corresponding diagnosis based on the image finding. After a diagnosis is determined, the workflow determines the level of retinopathy present based on the diagnosis ID, and assigns a treatment identification (ID) number. The treatment ID determines the treatment plan and whether or not the patient should be evaluated for laser treatment.

The recommendation module 240 may determine that the best management/treatment option for the patient is to schedule a follow-up visit because the current size and distribution of any lesions is not a significant medical concern. In another example, the recommendation module 240 may determine that the patient should visit a specialist within a few weeks because the lesions are of significant medical concern and require immediate attention. In yet another example, the recommendation module 240 may determine that no treatment action is required at this time because there are no lesions present based on the user inputs. The information related to a diagnosis, determined by the recommendation module 240, includes information that a diagnosis is unascertainable because the retina image is ungradable by mere observation in one or more categories of features. The information related to a treatment recommendation includes information relating to a follow-up visit recommendation, a referral information, a recommendation to evaluate for laser treatment, and the like.

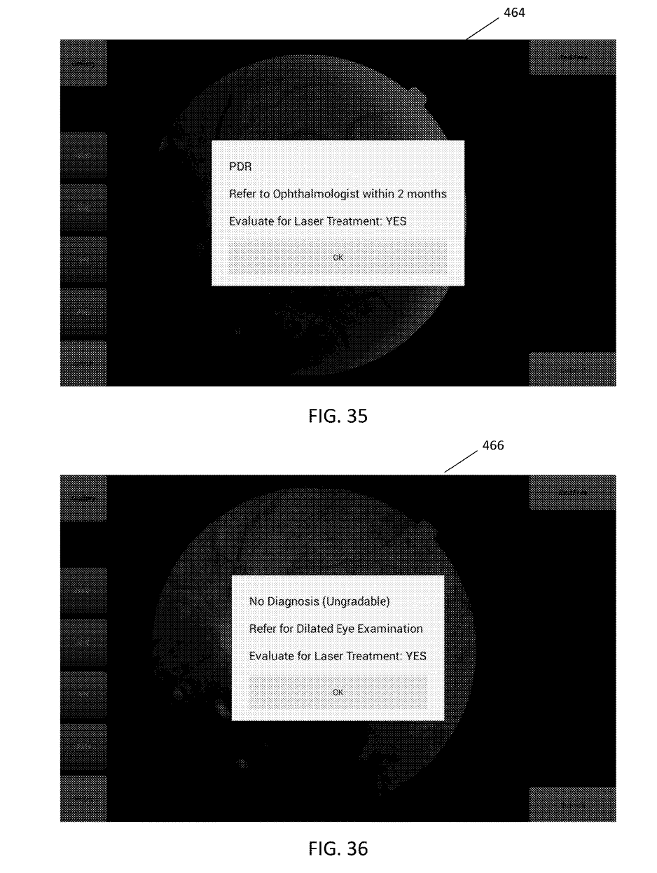

At block 314, the graphical user interface module 210 displays the information related to the diagnosis and treatment recommendation in the user interface, as shown in screen 464 of FIG. 35. In this example, screen 464 displays the diagnosis information as ‘PDR,’ and treatment information as ‘Refer to ophthalmologist within 2 months,’ and ‘Evaluate for Laser Treatment: YES.’ As discussed above, if any of the categories of features is ungradable, then the diagnosis information will indicate that by displaying “No Diagnosis (Ungradable)”, as shown in screen 466 of FIG. 36. In that case, the treatment recommendation information includes a referral for a dilated eye examination, and screen 466 displays “Refer for Dilated Eye Examination.” Further detail regarding a determination of findings, diagnosis information and a treatment recommendation based on selected features is provided below in the section entitled Exemplary Workflow Logic.

In an example embodiment, the storage module 250 may store the user inputs as data associated with the user's login information, or as associated with a patient identification number, or as associated with both. In some embodiments, the stored data forms an electronic medical record for the patient.

In some embodiments, the stored data, as an electronic medical record, may be transmitted from one device to another, for example, from a remotely located user device to a medical provider's or diagnosing physician's device.

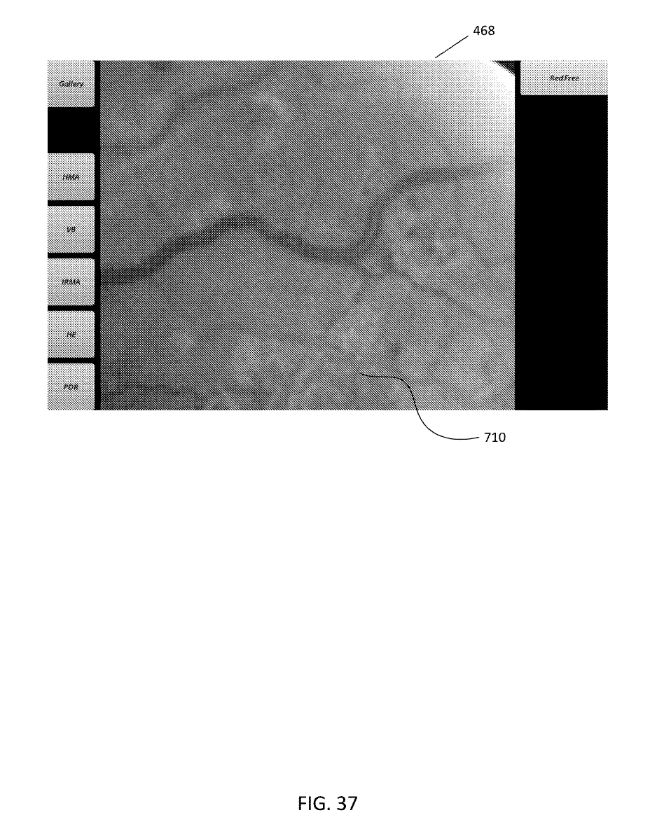

In this manner, the user selections indicate a grading of the various features present in the retina image via the various input controls in the user interface. This grading is provided by the user based on his or her observation of the displayed retina image. The user inputs his or her observations via the various buttons on the user interface. In an example embodiment, the user can perform various functions with respect to the retina image, for example, zoom or pan. FIG. 37 is an example screen 468 where the user zoomed into a feature of the retina image 710. The user can use his or her fingers to zoom-in or zoom-out on a touch-screen device, for example, by touching the screen with two fingers and changing the spacing between the two fingers. Similarly, the user can use his or her fingers to translate the image, for example, buy touching the screen with a finger and sliding the finger. In some embodiments, the zoom and translation features are enabled and available whenever an image of the retina is displayed on the screen. In some embodiments, when a user is zooming or panning the retina image, the buttons 505, 510, 515, 520, 525, 610 and 620 may temporarily disappear from the user interface screen. For example, the mobile device may be programmed to detect motion via the user's fingers that indicate a zooming or panning functions, and in response to detecting such motion, the mobile device is programmed to make the buttons 505, 510, 515, 520, 525, 610 and 620 disappear, fade, or withdraw into the outer edges of the user interface screen. The mobile device is further programmed to detect that the motion indicating zooming or panning has stopped, and in response, the mobile device is programmed to make the buttons 505, 510, 515, 520, 525, 610 and 620 reappear on the user interface screen. The mechanism of fading and reappearing the buttons during a zoom or pan function, enables the user to view a larger portion of the retina image. In an example embodiment, the control buttons on the right side and left side of the user interface screen take up approximately 20% of the user interface screen, and limit the user's view of the retina image. The fading or withdrawing of the control buttons aids the user to view the retina image properly and allows the user to investigate lesions in the retina image. Such zooming and panning may be particularly useful when marking retinal features as described below with respect to FIG. 40.

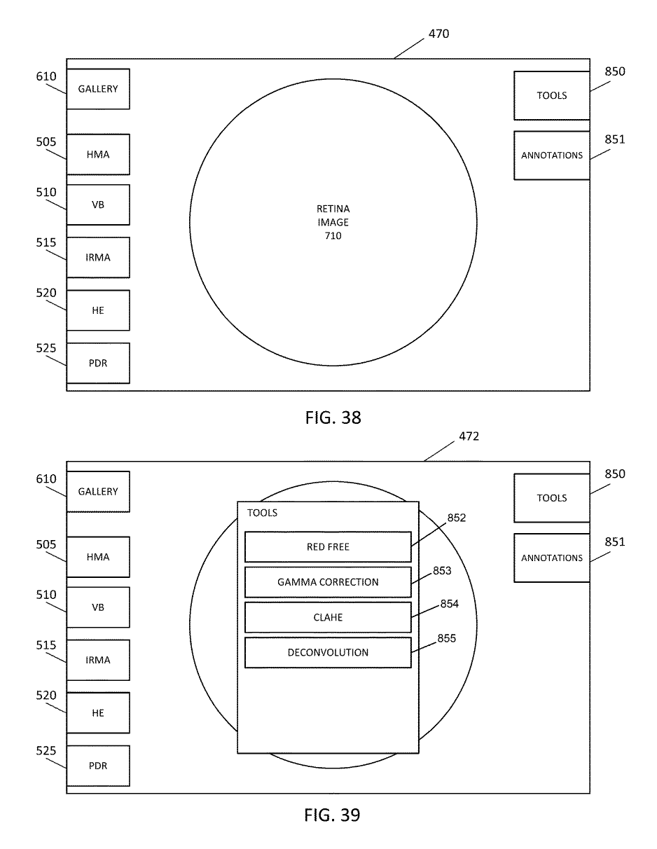

FIG. 38 is a schematic illustrating an example user interface screen 470, according to another example embodiment. As shown in FIG. 38 and as described above, the user interface screen 470 includes retina image 710, and buttons 505, 510, 515, 520, 525, and 610. In an example embodiment, the user interface screen 470 includes button 850, which upon selection provides access to image restoration tools, and button 851, which upon selection provides access to annotation tools. In some embodiments, the buttons 850 and 851 are located below button 620 for viewing an red-free image (see FIG. 7), and the user interface screen 470 may include feature-specific buttons 801-831 (see FIGS. 8, 11, 14, 17, 22, 25, 28, 31) based upon selection of a button 505, 510, 515, 520, or 525, as described above in relation with FIGS. 8-32.

FIG. 39 is a schematic illustrating an example user interface screen 472 displaying the image restoration tools available according to an example embodiment. In response to selecting button 850, the user interface screen 472 displays a dialog box including control buttons that provide access to various image restoration tools. For example, as shown in FIG. 39, the dialog box includes control buttons 852, 853, 854, and 855.

Button 852 can be toggled on or off to view a red-free image. When button 852 is toggled on, the retina image is displayed as a grayscale image, without any visible color. When button 852 is toggled on, the retina image is displayed with all color components present. In some embodiments, the button 852 may be a circular icon filled with either red or green color, and green or red border respectively. An icon with red color fill and green border may display a retina image with all color components present. An icon with green color fill and red border may display a retina image as a grayscale image, without any visible color. Toggling the button switches the icon from a red fill and green border icon to a green fill and red border icon.

Button 853 enables a user to perform gamma correction to a retina image. Gamma correction is a nonlinear operation used to adjust the luminance values in an image. In an example embodiment, a user may perform gamma correction on the retina image for nominal preset gamma values of 0.7, 1.0, or 1.3. Toggling the button 853 enables a user to select between the different gamma values of 0.7, 1.0, and 1.3. Different values may be used in practice, depending upon retinal image characteristics, ambient lighting, display screen characteristics, etc.

Button 854 enables a user to adjust the visibility of features in a retina image via the contrast limited adaptive histogram equalization (CLAHE) method. Button 854 can be toggled between high contrast, low contrast, and off setting. In alternative embodiments, any other suitable methods may be used to enable a user to adjust the contrast in a retina image.

Button 855 enables a user to adjust the sharpness of a retina image and/or to partially or fully remove noise or distortion caused in the image by the image capturing instrument (such as a camera, a mobile device camera, a microscope, and the like) via a deconvolution method. Button 855 can be toggled between high sharpness, low sharpness, and off setting. In alternative embodiments, any other suitable methods may be used to enable a user to adjust the sharpness in a retina image.

A user can select/toggle buttons 852-855 to obtain an optimal retina image for evaluation. The image restoration settings selected by the user persist through the evaluation and grading workflow, as the user selects the various category buttons 505, 510, 515, 520, and 525. The user can also change the image restoration settings during the workflow to adjust the image for a selected category.

In some embodiments, rather than including a tools button 850 to access image restoration tools, the buttons 852-855 corresponding to image restoration tools may be displayed as icons in the user interface screen, for example, along the top portion of the screen. The user may toggle the icon button to adjust the various features of the retina image, as described in relation to FIG. 39.

FIG. 40 is a schematic illustrating an example user interface screen 474 including markers on the retina image according to an example embodiment. Upon selection of button 851, a user is able to annotate the retina image by placing markers on the image as shown in FIG. 40. A user can tap (with his or her finger or a stylus) the touch-screen interface of his or her device to place a marker on the retina image. A user can select an existing marker, and drag it to move it to another position on the retina image. A user can erase an existing marker by double-tapping the marker (that is two quick sequential taps on the touch-screen interface). A user can place overlapping markers when closely-spaced lesions are present in the retina image. A user can also write notes, typically using a stylus, on the retina image.

In some embodiments, the markers may be different types of icons. For example, as shown in FIG. 40, the marker is illustrated as a triangle icon with text that indicates the type of lesion observed in the retina image. As another example, the marker may be a cross-hair icon with an empty center. In some embodiments, the cross-hair icon may be used as a marker for point-type lesions, generally observed by a user under the NPDR category (accessible via button 550 described above). As yet another example, the marker may be a circle. A user may be able to adjust the size of the circle marker, using his or her finger, to indicate a large or small lesion in the retina image. This type of circle marker may be used to indicate lesions in the PDR category, which is accessible via button 525 described above.

In some embodiments, the annotations (markers and notes) made by a user are stored with respect to a selected category. The annotations made while a category is selected, disappear from the user interface screen when the user selects another category. If the user returns to the previously selected category where he or she entered annotations, the stored annotations are re-displayed on the user interface screen. In some embodiments, multiple markers indicating different lesion types may be displayed simultaneously on the user interface screen for a user to easily reference the different types of lesions present in the retina image. The markers zoom and pan along with the retina image.

In some embodiments, all markings on the retinal image are recorded and stored in accordance with the DICOM standard.

The retinopathy evaluation system guides the user through the workflow by determining the subsequent steps based on the previous user input. The retinopathy evaluation system includes a workflow that determines the steps or criteria the user performs to receive a diagnosis or treatment recommendation. The application leads the user through a set of so-called decision points (e.g., categories of features) that helps in evaluating the retina image. The user can select from a number of outcomes of a decision point (e.g., set of features associated with a selected category), and selection of the outcomes (e.g., features for various decision points) determines the treatment recommendation or diagnosis. The manner in which the decision points (categories of features) are presented to the user allows the user to navigate in a particular order so that all information is properly captured, and a recommendation can be provided. In an example embodiment, the user is presented with decision points on the left hand side of the screen, as shown by buttons 505, 510, 515, 520 in FIG. 6. Selecting a decision point, displays a set of possible outcomes, as shown by buttons 801-806 in FIG. 8, on the right hand side of the screen. The user can select one of buttons 801-806 that represents the appropriate observation for that category based on the user viewing the retinal image. The user continues by selecting the next decision point (e.g., any other category on the left hand side of the screen), which displays corresponding outcomes (e.g., set of features) on the right hand side for the user to select from. The user again selects an outcome that represents his/her observation of the retina image. In this way, the user grades the lesions in the retinal image, and inputs his/her observation through the easy-to-use user interface of the retinopathy evaluation system. Based on the recommendation or diagnosis displayed by the retinopathy evaluation system, the user can instruct the patient to take appropriate steps.

In some embodiments, the client device 110, 115, 120, or 125 may be capable of operatively connecting to an external device or system capable of obtaining a retina image suitable for retinopathy evaluation or analysis. Such devices and systems include, but are not limited to fundus photography systems, optical coherence tomography systems, ultra wide field retinal imaging systems, and the like. The retinopathy workflow, evaluation and grading application may be configured to allow a user to take images of a retina using a suitable imaging device or system. These images may satisfy the “gold standard” 7 overlapping images approach to by using a single, nominal 90×60 degree field of view centered in the fovea.

In an example embodiment, a user can use the retinopathy workflow, evaluation and grading application for patient education (e.g., to show a patient his or her retina images, diagnosis and treatment progression). In this embodiment, the user interface is similar to the screens shown in FIGS. 5-37, however, the user may not be able to alter or change the information displayed on the screen. The user can only select buttons 505, 510, 515, 520, 525, 530, 535, 540, 545, and 550 to view the features selected for each categories of features, but he or she cannot select any of buttons 801-831 for the features. The appropriate feature buttons 801-831 are already highlighted based on information previously entered. The user can show these features to the patient, so that the patient can understand the reasons and physical manifestations associated with for his or her diagnosis. The user can also show a patient images from the patient's previous visit and images from the patient's present visit, so as to compare the progression of the disease or progress of the treatment.

Exemplary Workflow Logic

Tables 1 and 2 include an exemplary workflow included in the retinopathy evaluation system that drives the logic to determine diagnosis information and a treatment plan, in accordance with some embodiments. In an example embodiment, the workflow embodies the Early Treatment Diabetic Retinopathy Study (ETDRS) standard protocols for evaluating diabetic retinopathy. Table 1 below illustrates example workflow logic for determining image findings and diagnosis information based on the features selected by the user. The parameters shown (e.g., HMA: <5 HMA or HE: >500μ) for evaluating and grading each feature within Table 1, Category: Selected Feature column are examples, and the specific range for the features may change according to local clinical workflow and practice.

TABLE 1
Workflow Logic for Determining a Diagnosis

| Category: | Image Findings based on | Diagnosis | |
|---|---|---|---|
| Selected Feature | Selected Feature | Id. | Diagnosis |
| All: clear | No findings | 1 | No Evidence of |
| | | | Retinopathy |
| HMA: <5 HMA | Microaneurysms only | 2 | Mild NPDR |
| HMA: <2A, 1-4Q | >Mild and <Severe | 3 | Moderate NPDR |
| HMA: >2A, 1-3Q | Findings | | |
| HMA: >2A, 4Q | (>20 HMA in all | 4 | Severe NPDR |
| VB: >6B, 2-4Q | quadrants) or (Venous | | |
| or higher | Beading in 2+ quadrants) | | |
| IRMA: <8A, 1- | or (IRMA in 1+ quadrant) | | |
| 3Q | | | |
| IRMA: >8A, 1Q | | | |
| or <8A, 1-4Q | | | |
| NVD: present | Neovascularization | 5 | PDR |
| NVE: present | or Vitreous | | |
| VH: present | or Preretinal Hemorrhage | | |
| PVH: present | | | |
| HE: clear | No Hard Exudates | 6 | No Diabetic Macular |
| | | | Edema |
| HE: >500 | Hard Exudates present >500μ | 7 | Diabetic Macular Edema |
| | from center of the | | likely |
| | macula | | |
| HE: <500 | Hard Exudates present <500μ | 8 | Clinically Significant |
| | from center of the | | Diabetic Macular Edema |
| | macula | | likely |
| Any: ungradable | Ungradable | 11 | No Diagnosis |

Table 2 below illustrates example workflow logic for determining a treatment recommendation based on a diagnosis.

TABLE 2
Workflow Logic for Determining a Treatment Recommendation

| Basis for | Level of | | | Evaluate |
|---|---|---|---|---|
| Treatment | Diabetic | Recommendation | | for Laser |
| Recommendation | Retinopathy | Id. | Recommendation | Treatment? |
| Clean retina | No Evidence of | 1 | Reevaluate in 1 | No |
| | Retinopathy | | year | |
| Diagnosis 2 and 6 | Mild NPDR | 2 | Reevaluate in 6 | No |
| | without Diabetic | | months | |
| | Macular Edema | | | |
| Diagnosis 2 and 7 | Mild NPDR | 3 | Refer to | No |
| | with risk of | | ophthalmologist | |
| | Diabetic | | within 4 months | |
| | Macular Edema | | | |
| Diagnosis 2 and 8 | Mild NPDR | 4 | Refer to | Yes |
| | with risk of | | ophthalmologist | |
| | Clinically | | within 2 months | |
| | Significant | | | |
| | Macular Edema | | | |
| Diagnosis 3 and 6 | Moderate NPDR | 5 | Refer to | No |
| | without Diabetic | | ophthalmologist | |
| | Macular Edema | | within 6 months | |
| Diagnosis 3 and 7 | Moderate NPDR | 6 | Refer to | No |
| | with risk of | | ophthalmologist | |
| | Diabetic | | within 4 months | |
| | Macular Edema | | | |
| Diagnosis 3 and 8 | Moderate NPDR | 7 | Refer to | Yes |
| | with risk | | ophthalmologist | |
| | Clinically | | within 2 months | |
| | Significant | | | |
| | Macular Edema | | | |
| Diagnosis 4 | Severe NPDR | 8 | Refer to | Yes |
| | | | ophthalmologist | |
| | | | within 2 months | |
| Diagnosis 5 | PDR | 9 | Refer to | Yes |
| | | | ophthalmologist | |
| | | | within 2 months | |
| Diagnosis 11 | Ungradable | 10 | Refer for dilated | Yes |
| | | | eye examination | |

For example, if all of the selected features are clear for an image that indicates that no findings were observed in the retina image. In this case, the diagnosis ID 1 is assigned which corresponds to the diagnosis “No Evidence of Retinopathy.” In the case where the user observes less than 5 hemorrhages and microaneurysms and selects the corresponding feature (HMA<5 HMA), the image findings indicate microaneurysms only. This is assigned the diagnosis ID 2 which corresponds to the diagnosis “Mild NPDR.” In the next case where the user observes hemorrhages and microaneurysms that are less than <2A in one to four quadrants of the retina (selected feature HMA <2A, 1-4Q), then the image findings indicate more than mild but less than severe findings. This is assigned the diagnosis ID 3 corresponding to diagnosis “Moderate NPDR.” Where the user observes hemorrhages and microaneurysms that are more than >2A in one to three quadrants of the retina (selected feature HMA>2A, 1-3Q), then the image findings indicate more than mild but less than severe Findings. This is also assigned the diagnosis ID 3 corresponding to diagnosis “Moderate NPDR.”

In the case where the user observes hemorrhages and microaneurysms that are more than >2A in four quadrants of the retina (selected feature HMA>2A, 4Q), venous beading more than 6B in two to four quadrants or higher (selected feature VB>6B, 2-4Q or higher), intraretinal microvascular abnormalities less than 8A in one to three quadrants (selected feature IRMA<8A, 1-3Q), or intraretinal microvascular abnormalities more than 8A in one quadrant or less than 8A in one to four quadrants (selected feature IRMA>8A, 1Q or <8A, 1-4Q), then the image findings indicate >20 HMA in all quadrants or venous beading in 2+ quadrants or intraretinal microvascular abnormalities in 1+ quadrant. This case is assigned the diagnosis ID 4 corresponding to diagnosis “Severe NPDR.”

In the case where the user observes new vessels on the disc (NVD: present), new vessels elsewhere (NVE: present), vitreous hemorrhage (VH present), or preretinal vitreous hemorrhage (PVH: present), then image findings indicate neovascularization or vitreous or preretinal hemorrhage. This case is assigned the diagnosis ID 5 corresponding to diagnosis “PDR.”

In the case where the user observes no hard exudates (HE: clear), then image findings indicate no hard exudates. This case is assigned diagnosis ID 6 corresponding to diagnosis “No Diabetic Macular Edema.” In the case where the user observes hard exudates more than 500μ from the center of the fovea (HE: >500), then image findings indicate hard exudates present >500μ from center of the macula. This case is assigned diagnosis ID 7 corresponding to “Diabetic Macular Edema likely.” In the case where the user observes hard exudates less than 500μ from the center of the fovea (HE: <500) then image findings indicate hard exudates present <500μ from center of the macula. This case is assigned diagnosis ID 8 corresponding to “Clinically Significant Diabetic Macular Edema likely.” In the case where the user observes any ungradable features (selected feature Ungradable) then image findings indicate ungradable. This case is assigned diagnosis ID 11 corresponding to “No Diagnosis.” Even though ID 11 is not an actual diagnosis, the determination that no diagnosis is available due to an ungradable retina image is information regarding a diagnosis.

Once the diagnosis is determined based on the selected features, a level of diabetic retinopathy and a treatment recommendation are determined. In the case where the retina is clean (i.e. there is no evidence of retinopathy), treatment ID 1 is assigned corresponding to “Reevaluate in 1 year” and do not evaluate for laser treatment. In the case where the retina presents mild NPDR without diabetic macular edema (diagnosis IDs 2 and 6), the treatment ID 2 is assigned corresponding to “Reevaluate in 6 months” and do not evaluate for laser treatment. Where the retina presents mild NPDR with risk of diabetic macular edema (diagnosis IDs 2 and 7), the treatment ID 3 is assigned corresponding to “Refer to ophthalmologist within 4 months” and do not evaluate for laser treatment. Where the retina presents mild NPDR with risk of clinically significant diabetic macular edema (diagnosis IDs 2 and 8), the treatment ID 4 is assigned corresponding to “Refer to ophthalmologist within 2 months” and evaluate for laser treatment. In the case where the retina presents moderate NPDR without diabetic macular edema (diagnosis IDs 3 and 6), the treatment ID 5 is assigned corresponding to “Refer to ophthalmologist within 6 months” and do not evaluate for laser treatment. In the case where the retina presents moderate NPDR with risk of diabetic macular edema (diagnosis IDs 3 and 7), the treatment ID 6 is assigned corresponding to “Refer to ophthalmologist within 4 months” and do not evaluate for laser treatment. Where the retina presents moderate NPDR with risk of clinically significant diabetic macular edema (diagnosis IDs 3 and 8), the treatment ID 7 is assigned corresponding to “Refer to ophthalmologist within 2 months” and evaluate for laser treatment. Where the retina presents severe NPDR (diagnosis IDs 4), the treatment ID 8 is assigned corresponding to “Refer to ophthalmologist within 2 months” and evaluate for laser treatment. Where the retina presents PDR (diagnosis IDs 5), the treatment ID 9 is assigned corresponding to “Refer to ophthalmologist within 2 months” and evaluate for laser treatment. In the case where the retina is ungradable (diagnosis ID 11), the treatment ID 10 is assigned corresponding to “Refer for Dilated eye examination” and evaluate for laser treatment.

In this manner, the example workflow logic above embodies the standards of ETDRS. In other embodiments, workflow logic that embodies other standards for other types of retinopathies can be used within the retinopathy evaluation system.

Example Alternative System Embodiment

In some embodiments, a mobile device may be only a portion of a networked retinopathy evaluation system. In some embodiments, a mobile device that embodies a retinopathy evaluation system may interact with other devices, or resources via a network. FIG. 38 illustrates a network diagram depicting a system 100 for a retinopathy evaluation system according to an example embodiment. The system 100 can include a network 105, a client device 110, a client device 115, a client device 120, a client device 125, a database(s) 130, a server 135, and a database server(s) 140. Each of the client devices 110, 115, 120, 125, database(s) 130, server 135, and database server(s) 140 is in communication with the network 105.

In an example embodiment, one or more portions of network 105 may be an ad hoc network, an intranet, an extranet, a virtual private network (VPN), a local area network (LAN), a wireless LAN (WLAN), a wide area network (WAN), a wireless wide area network (WWAN), a metropolitan area network (MAN), a portion of the Internet, a portion of the Public Switched Telephone Network (PSTN), a cellular telephone network, a wireless network, a WiFi network, a WiMax network, any other type of network, or a combination of two or more such networks.

In an example embodiment, the client device 110, 115, 120, or 125 is a mobile client device. Examples of a mobile client device includes, but are not limited to, hand-held devices, wireless devices, portable devices, wearable computers, cellular or mobile phones, portable digital assistants (PDAs), smartphones, tablets, ultrabooks, netbooks, multi-processor systems, microprocessor-based or programmable consumer electronics, mini-computers, smart watches, and the like. In alternative embodiments, the client device 110, 115, 120, 125 may comprise work stations, personal computers, general purpose computers, Internet appliances, laptops, desktops, multi-processor systems, set-top boxes, network PCs, vehicle installed computer systems, and the like. Each of client devices 110, 115, 120, 125 may connect to network 105 via a wired or wireless connection. Each of client devices 110, 115, 120, 125 may include one or more applications (also referred to as “apps”) such as, but not limited to, a web browser, messaging application, electronic mail (email) application, notification application, photo or imaging application, a retinopathy application described herein, and the like. In some embodiments, the retinopathy application included in any of the client devices 110, 115, 120, 125 may be configured to locally provide a user interface, locally perform the functionalities described herein, and communicate with network 105, on an as-needed basis, for acquiring data not locally available or for transferring data to a device or component connected to the network 105 (transfer or send data to other user's devices so that they may view the results of the retinopathy diagnosis).

In some embodiments, information created on the user's device (e.g., various findings, notes, a diagnosis and treatment plan) may be communicated to the patient's mobile device and form part of a portable medical record. In some embodiments, all or part of a portable medical record on a patient's device may be uploaded to the user's mobile device.

In some embodiments, a certain amount of data and/or certain type(s) of data are transmitted from any one of device 110, 115, 120, 125 to any other one of device 110, 115, 120, 125 based on the communication modality. For example, for low bandwidth settings, fundamental basic data, consisting primarily of text, may be transmitted. Such basic data may include biometric identifiers (facial, iris and retinal measurements), dates, and locations for prior treatment provided, attending physician, and the like. For higher bandwidth settings, more detailed data may be transmitted, such as, medical test records, biometric and diagnostic images, and the like. Biometric identifiers is generally transmitted to ensure that the medical record data selected by a user corresponds to a specific patient that may be present for evaluation. The bandwidth of the communication modality may be verified via test transmissions between the devices. The devices 110, 115, 120, 125, may be capable of employing various communication modalities such as NFC, Bluetooth, 2G mobile network, 3G mobile network, 4G mobile network, and others. In some embodiments, various encryption and data security protocols are implemented when storing and transmitting data. For example, electronic medical record data may be stored on a patient's device and transmitted to a medical provider's device or computer system. The amount and type of medical record data transmitted may be determined on the available communication modality.

In an example embodiment, the client device 110, 115, 120, 125 may process user input, determine the subsequent steps in the workflow, and prompt the user to follow those steps. The client device may determine a treatment recommendation and/or diagnosis based on the user input to the workflow. The client device 110, 115, 120, 125 may display the treatment recommendation and/or diagnosis to the user. Then when a network connection is available, the client devices 110, 115, 120, 125 may upload the treatment recommendation and diagnosis to a database, and store the data as corresponding to the user input, the user, and/or a patient identification number, thus, making it available for download by other authorized devices.

In other embodiments, the retinopathy application may be included on the client device 110, 115, 120, 125, and the server 135 performs the functionalities described herein. The server 135 may process and store user input received on the client devices 110, 115, 120, 125, and determine the subsequent steps based on the user input, and prompt the user to follow the steps. The server 135 may also determine a treatment recommendation and/or diagnosis based on the user input, and cause the client devices 110, 115,120, 125 to display the treatment recommendation and/or diagnosis. The server 135 may store the treatment recommendation and diagnosis as corresponding to the user input, the user, and/or a patient identification number, and make it available for download by other authorized devices.

In another embodiment, the retinopathy application included in any of the client devices 110, 115, 120, 125 may be configured to locally perform some of the functionalities described herein, while the server 135 performs the other functionalities described herein. For example, the client device 110, 115, 120, 125 may process and store user input received thereon. The client device 110, 115, 120, 125 may determine the subsequent steps in the workflow based on the user input, and prompt the user to follow those steps. The server 135 may determine a treatment recommendation and/or diagnosis based on the user input. The client device 110, 115, 120, 125 may display the treatment recommendation and/or diagnosis to the user. The server 135 may store the treatment recommendation and diagnosis as corresponding to the user input, the user, and/or a patient identification number, and make it available for download by other authorized devices.

In some embodiments, each of the database(s) 130, server 135, and database server(s) 140 is connected to the network 105 via a wired connection. Alternatively, one or more of the database(s) 130, server 135, or database server(s) 140 may be connected to the network 105 via a wireless connection. Although not shown, database server(s) 140 can be (directly) connected to database(s) 130, or server 135 can be (directly) connected to the database server(s) 140 and/or database(s) 130. Server 135 comprises one or more computers or processors configured to communicate with client devices 110, 115, 120, 125 via network 105. Server 135 hosts one or more applications or websites accessed by client devices 110, 115, 120, and 125 and/or facilitates access to the content of database(s) 130. Database server(s) 140 comprises one or more computers or processors configured to facilitate access to the content of database(s) 130. Database(s) 130 comprise one or more storage devices for storing data and/or instructions for use by server 135, database server(s) 140, and/or client devices 110, 115, 120, 125. Database(s) 130, server 135, and/or database server(s) 140 may be located at one or more geographically distributed locations from each other or from client devices 110, 115, 120, 125. Alternatively, database(s) 130 may be included within server 135 or database server(s) 140.

In an alternative embodiment, the retinopathy workflow, evaluation, and grading application may be a web-based application that can be accessed on client devices 110, 115, 120, 125 via a web-browser application.

Certain embodiments are described herein as including logic or a number of components, modules, or mechanisms. Modules may constitute either software modules (e.g., code embodied on a machine-readable medium or in a transmission signal) or hardware modules. A hardware module is a tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA), an application-specific integrated circuit (ASIC), or a Graphics Processing Unit (GPU)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired) or temporarily configured (e.g., programmed) to operate in a certain manner and/or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., APIs).

Example embodiments may be implemented in digital electronic circuitry, or in computer hardware, firmware, software, or in combinations of them. Example embodiments may be implemented using a computer program product, for example, a computer program tangibly embodied in an information carrier, for example, in a machine-readable medium for execution by, or to control the operation of, data processing apparatus, for example, a programmable processor, a computer, or multiple computers.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network.

In example embodiments, operations may be performed by one or more programmable processors executing a computer program to perform functions by operating on input data and generating output. Method operations can also be performed by, and apparatus of example embodiments may be implemented as, special purpose logic circuitry (e.g., a FPGA or an ASIC).

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., machine) and software architectures that may be deployed, in various example embodiments.

FIG. 39 is a block diagram of machine in the example form of a computer system 900 (e.g., a mobile device) within which instructions, for causing the machine (e.g., client device 110, 115, 120, 125; server 135; database server(s) 140; database(s) 130) to perform any one or more of the methodologies discussed herein, may be executed. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a tablet PC, a set-top box (STB), a PDA, a cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 900 includes a processor 902 (e.g., a central processing unit (CPU), a multi-core processor, and/or a graphics processing unit (GPU)), a main memory 904 and a static memory 906, which communicate with each other via a bus 908. The computer system 900 may further include a video display unit 910 (e.g., a liquid crystal display (LCD), a touch screen, or a cathode ray tube (CRT)). The computer system 900 also includes an alphanumeric input device 912 (e.g., a physical or virtual keyboard), a user interface (UI) navigation device 914 (e.g., a mouse), a disk drive unit 916, a signal generation device 918 (e.g., a speaker) and a network interface device 920.

The disk drive unit 916 includes a machine-readable medium 922 on which is stored one or more sets of instructions and data structures (e.g., software) 924 embodying or used by any one or more of the methodologies or functions described herein. The instructions 924 may also reside, completely or at least partially, within the main memory 904, static memory 906, and/or within the processor 902 during execution thereof by the computer system 900, the main memory 904 and the processor 902 also constituting machine-readable media.

While the machine-readable medium 922 is shown in an example embodiment to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding or carrying instructions for execution by the machine and that cause the machine to perform any one or more of the methodologies of the present invention, or that is capable of storing, encoding or carrying data structures used by or associated with such instructions. The term “machine-readable medium” shall accordingly be taken to include, but not be limited to, solid-state memories, and optical and magnetic media. Specific examples of machine-readable media include non-volatile memory, including by way of example, semiconductor memory devices (e.g., Erasable Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 924 may further be transmitted or received over a communications network 926 using a transmission medium. The instructions 924 may be transmitted using the network interface device 920 and any one of a number of well-known transfer protocols (e.g., HTTP). Examples of communication networks include a LAN, a WAN, the Internet, mobile telephone networks, Plain Old Telephone (POTS) networks, and wireless data networks (e.g., Wi-Fi and WiMax networks). The term “transmission medium” shall be taken to include any intangible medium that is capable of storing, encoding or carrying instructions for execution by the machine, and includes digital or analog communications signals or other intangible media to facilitate communication of such software.

Although the present invention has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense.

It will be appreciated that, for clarity purposes, the above description describes some embodiments with reference to different functional units or processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although an embodiment has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be used and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended; that is, a system, device, article, or process that includes elements in addition to those listed after such a term in a claim are still deemed to fall within the scope of that claim. Moreover, in the following claims, the terms “first,” “second,” and “third” and so forth are used merely as labels, and are not intended to impose numerical requirements on their objects.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

1. A computer-implemented method for guiding a user through a retinopathy evaluation of a retina image on a client mobile device having a processor, the method comprising:
displaying the retina image in a user interface on the client mobile device;
displaying a first plurality of user input controls in the user interface, the first plurality of user input controls representing a plurality of categories of features related to retinopathy evaluation;
receiving a selection of a first user input control from the first plurality of user input controls corresponding to a first selected category of features from the plurality of categories of features, the selection of the first user input control received via a touch-screen interface of the client mobile device;
in response to receiving the selection of the first user input control, displaying a second plurality of user input controls in the user interface, the second plurality of user input controls representing a first set of features associated with the first selected category of features;
receiving a selection of a second user input control from the second plurality of user input controls corresponding to a first selected feature from the first set of features indicating a first feature appearing in the retina image;
determining one or more findings based on, at least, the first selected feature using the processor of the client mobile device;
determining information related to a diagnosis based on the one or more findings using the processor of the client mobile device; and
displaying the information related to the diagnosis on the user interface.

2. The method of claim 1, further comprising:
receiving a selection of a third user input control from the first plurality of user input controls corresponding to a second selected category of features from the plurality of categories of features;
in response to receiving the selection of the third user input control, displaying a third plurality of user input controls in the user interface, the third plurality of user input controls representing a second set of features associated with the second selected category of features; and
receiving a selection of a fourth user input control from the third plurality of user input controls corresponding to a second selected feature from the second set of features indicating a second feature appearing in the retina image;
wherein the determining one or more findings is based on, at least, the first selected feature and the second selected feature.

3. The method of claim 2, further comprising:
receiving a selection of a fifth user input control from the first plurality of user input controls corresponding to a third selected category of features from the plurality of categories of features;
in response to receiving the selection of the fifth user input control, displaying a fourth plurality of user input controls in the user interface, the fourth plurality of user input controls representing a third set of features associated with the third selected category of features; and
receiving a selection of a sixth user input control from the fourth plurality of user input controls corresponding to a third selected feature from the third set of features indicating a third feature appearing in the retina image;
wherein the determining one or more findings is based on, at least, the first selected feature, the second selected feature, and the third selected feature.

4. The method of claim 1, further comprising enabling the user to view the retina image in the user interface at the same time as the first and second plurality of user input controls.

5. The method of claim 1, further comprising displaying a recommendation for follow up action based on the information for the diagnosis.

6. The method of claim 1, further comprising displaying treatment information based on the information for the diagnosis.

7. The method of claim 1, wherein the user input controls are buttons.

8. The method of claim 7, further comprising indicating the selection of the first user input control by changing a color of corresponding button.

9. The method of claim 1, wherein the determining of the one or more findings is based on receiving a selection of a feature for each category of features of the plurality of categories of features.

10. The method of claim 1, further comprising, displaying a summary of any selected features in the user interface on the client device.

11. The method of claim 1, wherein the plurality of categories of features includes three or more of hemorrhages and microaneurysms, venous beading, intraretinal microvascular abnormalities, hard exudates, new vessels on the disc, new vessels elsewhere, vitreous hemorrhage, and preretinal vitreous hemorrhage.

12. The method of claim 1, wherein the first plurality of user input controls are displayed on a first side portion of a screen of the client mobile device, the second plurality of user input controls are displayed on a second side portion of the screen opposite the first side portion, and the retina image is displayed in a central portion of the screen.

13. The method of claim 1, further comprising:
displaying a plurality of retina images in the user interface;
receiving a selection of the retina image from the plurality of retina images; and
displaying the selected retina image in the user interface.

14. A computer-implemented method for confirming a retinopathy assessment of a retina image on a client mobile device having a processor, the method comprising:
displaying the retina image in a user interface on the client device;
displaying a first plurality of user input controls in the user interface, the first plurality of user input controls representing a plurality of categories of features related to the retinopathy assessment;
receiving a selection of a first user input control from the first plurality of user input controls corresponding to a first selected category of features from the plurality of categories of features, the selection of the first user input control received via a touch-screen interface of the client mobile device;
in response to receiving the selection of the first user input control, displaying a second plurality of user input controls in the user interface, the second plurality of user input controls representing a first set of features associated with the first selected category of features and including at least one highlighted user input control corresponding to a previously identified feature present in the retina image during the retinopathy assessment; and
displaying the retinopathy assessment on the user interface.

15. The method of claim 14, further comprising:
receiving a selection of a second user input control from the second plurality of user input controls corresponding to a feature not highlighted in the user interface;
determining one or more findings based on, at least, the first selected feature using the processor of the client mobile device; and
determining a revised retinopathy assessment based on the one or more findings using the processor of the client mobile device; and
tracking the selection of the second user input control and the revised retinopathy assessment as a correction to the retinopathy assessment.

16. The method of claim 14, further comprising:
receiving an indication via the user interface confirming the retinopathy assessment.

17. The method of claim 14, further comprising:
displaying a summary of the features contributing to the retinopathy assessment.

18. A computer-implemented method for educating a patient of a retinopathy assessment on a client mobile device having a processor, the method comprising:
displaying the retina image in a user interface on the client mobile device;
displaying a plurality of selectable categories of features relating to the retinopathy assessment;
receiving a selection of a category of features from the plurality of categories of features via a touch-screen interface of the client mobile device;
in response to receiving the selection of the category of features, displaying a set of features corresponding to the selected category of features, wherein at least one feature of the set of features is highlighted, the highlighted feature previously identified as present in the retina image during the retinopathy assessment; and
displaying previously assessed information related to a diagnosis based on the highlighted features.

19. The method of claim 18, further comprising displaying a summary of the selected features contributing to the retinopathy assessment.

20. (canceled)

21. A system for guiding a user through a retinopathy evaluation of a retina image on a client mobile device having a processor, the system comprising:
a display module configured to display the retina image in a user interface on the mobile client device, and a first plurality of user input controls in the user interface, the first plurality of user input controls representing a plurality of categories of features related to retinopathy evaluation;
an input module configured to receive a selection of a first user input control from the first plurality of user input controls corresponding to a first selected category of features from the plurality of categories of features, the selection of the first user input control received via a touch-screen interface of the client mobile device;
the display module further configured to display a second plurality of user input controls in the user interface, in response to receiving the selection of the first user input control, the second plurality of user input controls representing a first set of features associated with the first selected category of features;
the input module further configured to receive a selection of a second user input control from the second plurality of user input controls corresponding to a first selected feature from the first set of features indicating a first feature appearing in the retina image;
a processor-implemented recommendation module configured to determine one or more findings based on, at least, the first selected feature using the processor of the client mobile device, and determine information related to a diagnosis based on the one or more findings using the processor of the client mobile device; and
the display module further configured to display the information related to the diagnosis on the user interface.
